SEQUENCE LISTING (70 sequences)

SEQ ID NO: 1 - DNA, 1509 bp, Homo sapiens
  misc_feature (1)..(1509): FL-WASP
atgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggccctcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggccctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactga

SEQ ID NO: 2 - protein, 502 aa, Homo sapiens
  misc_feature (1)..(502): FL-WASP
MSGGPMGGRPGGRGAPAVQQNIPSTLLQDHENQRLFEMLGRKCLTLATAVVQLYLALPPGAEHWTKEHCGAVCFVKDNPQKSYFIRLYGLQAGRLLWEQELYSQLVYSTPTPFFHTFAGDDCQAGLNFADEDEAQAFRALVQEKIQKRNQRQSGDRRQLPPPPTPANEERRGGLPPLPLHPGGDQGGPPVGPLSLGLATVDIQNPDITSSRYRGLPAPGPSPADKKRSGKKKISKADIGAPSGFKHVSHVGWDPQNGFDVNNLDPDLRSLFSRAGISEAQLTDAETSKLIYDFIEDQGGLEAVRQEMRRQEPLPPPPPPSRGGNQLPRPPIVGGNKGRSGPLPPVPLGIAPPPPTPRGPPPPGRGGPPPPPPPATGRSGPLPPPPPGAGGPPMPPPPPPPPPPPSSGNGPAPPPLPPALVPAGGLAPGGGRGALLDQIRQGIQLNKTPGAPESSALQPPPQSSEGLVGALMHVMQKRSRAIHSSDEGEDQAGDEDEDDEWDD

SEQ ID NO: 3 - DNA, 1518 bp, Homo sapiens
  misc_feature (1)..(1518): FL-N-WASP
atgagctccgtccagcagcagccgccgccgccgcggagggtcaccaacgtggggtccctgttgctcaccccgcaggagaacgagtccctcttcactttcctcggcaagaaatgtgtgactatgtcttcagcagtggtgcagttatatgcagcagatcggaactgtatgtggtcaaagaagtgcagtggtgttgcttgtcttgttaaggacaatccacagagatctcattttttaagaatatttgacattaaggatgggaaactattgtgggaacaagagctatacaataactttgtatataatagtcctagaggatattttcatacctttgctggagatacttgtcaagttgctcttaattttgccaatgaagaagaagcaaaaaaatttcgaaaagcagttacagaccttttgggccgtcgacaaaggaaatctgagaaaagacgagatcccccaaatggtcctaatctacccatggctacagttgatataaaaaatccagaaatcacaacaaatagattttatggtccacaagtcaacaacatctcccataccaaagaaaagaagaagggaaaagctaaaaagaagagattaaccaagggagatataggaacaccaagcaatttccagcacattggacatgttggttgggatccaaatacaggctctgatctgaataatttggatccagaattgaagaatctttttgatatgtgtggaatcttagaggcacaacttaaagaaagagaaacattaaaagttatatatgactttattgaaaaaacaggaggtgttgaagctgttaaaaatgaactgcggaggcaagcaccaccacctccaccaccatcaaggggagggccacctcctcctcctccccctccacatagctcgggtcctcctcctcctcctgctaggggaagaggcgctcctcccccaccaccttcaagagctcccacagctgcacctccaccaccgcctccttccaggccaagtgtagaagtccctccaccaccgccaaataggatgtaccctcctccacctccagcccttccctcctcagcaccttcagggcctccaccaccacctccatctgtgttgggggtagggccagtggcaccacccccaccgcctccacctccacctcctcctgggccaccgcccccgcctggcctgccttctgatggggaccatcaggttccaactactgcaggaaacaaagcagctcttttagatcaaattagagagggtgctcagctaaaaaaagtggagcagaacagtcggccagtgtcctgctctggacgagatgcactgttagaccagatacgacagggtatccaactaaaatctgtggctgatggccaagagtctacaccaccaacacctgcacccacttcaggaattgtgggtgcattaatggaagtgatgcagaaaaggagcaaagccattcattcttcagatgaagatgaagatgaagatgatgaagaagattttgaggatgatgatgagtgggaagactga

SEQ ID NO: 4 - protein, 505 aa, Homo sapiens
  misc_feature (1)..(505): FL-N-WASP
MSSVQQQPPPPRRVTNVGSLLLTPQENESLFTFLGKKCVTMSSAVVQLYAADRNCMWSKKCSGVACLVKDNPQRSYFLRIFDIKDGKLLWEQELYNNFVYNSPRGYFHTFAGDTCQVALNFANEEEAKKFRKAVTDLLGRRQRKSEKRRDPPNGPNLPMATVDIKNPEITTNRFYGPQVNNISHTKEKKKGKAKKKRLTKADIGTPSNFQHIGHVGWDPNTGFDLNNLDPELKNLFDMCGISEAQLKDRETSKVIYDFIEKTGGVEAVKNELRRQAPPPPPPSRGGPPPPPPPPHNSGPPPPPARGRGAPPPPPSRAPTAAPPPPPPSRPSVAVPPPPPNRMYPPPPPALPSSAPSGPPPPPPSVLGVGPVAPPPPPPPPPPPGPPPPPGLPSDGDHQVPTTAGNKAALLDQIREGAQLKKVEQNSRPVSCSGRDALLDQIRQGIQLKSVADGQESTPPTPAPTSGIVGALMEVMQKRSKAIHSSDEDEDEDDEEDFEDDDEWED

SEQ ID NO: 5 - DNA, 225 bp, Homo sapiens
  misc_feature (1)..(225): WASP VCA domain
gggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactga

SEQ ID NO: 6 - protein, 74 aa, Homo sapiens
  misc_feature (1)..(74): WASP VCA domain
GGRGALLDQIRQGIQLNKTPGAPESSALQPPPQSSEGLVGALMHVMQKRSRAIHSSDEGEDQAGDEDEDDEWDD

SEQ ID NO: 7 - DNA, 345 bp, Homo sapiens
  misc_feature (1)..(345): N-WASP VCA domain
ccttctgatggggaccatcaggttccaactactgcaggaaacaaagcagctcttttagatcaaattagagagggtgctcagctaaaaaaagtggagcagaacagtcggccagtgtcctgctctggacgagatgcactgttagaccagatacgacagggtatccaactaaaatctgtggctgatggccaagagtctacaccaccaacacctgcacccacttcaggaattgtgggtgcattaatggaagtgatgcagaaaaggagcaaagccattcattcttcagatgaagatgaagatgaagatgatgaagaagattttgaggatgatgatgagtgggaagactag

SEQ ID NO: 8 - protein, 114 aa, Homo sapiens
  misc_feature (1)..(114): N-WASP VCA domain
PSDGDHQVPTTAGNKAALLDQIREGAQLKKVEQNSRPVSCSGRDALLDQIRQGIQLKSVADGQESTPPTPAPTSGIVGALMEVMQKRSKAIHSSDEDEDEDDEEDFEDDDEWED

SEQ ID NO: 9 - DNA, 1197 bp, Homo sapiens
  misc_feature (1)..(1197): 105WASP
cttgtctactccacccccacccccttcttccacaccttcgctggagatgactgccaagcggggctgaactttgcagacgaggacgaggcccaggccttccgggccctcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggccctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactag

SEQ ID NO: 10 - protein, 398 aa, Homo sapiens
  misc_feature (1)..(398): 105WASP
LVYSTPTPFFHTFAGDDCQAGLNFADEDEAQAFRALVQEKIQKRNQRQSGDRRQLPPPPTPANEERRGGLPPLPLHPGGDQGGPPVGPLSLGLATVDIQNPDITSSRYRGLPAPGPSPADKKRSGKKKISKADIGAPSGFKHVSHVGWDPQNGFDVNNLDPDLRSLFSRAGISEAQLTDAETSKLIYDFIEDQGGLEAVRQEMRRQEPLPPPPPPSRGGNQLPRPPIVGGNKGRSGPLPPVPLGIAPPPPTPRGPPPPGRGGPPPPPPPATGRSGPLPPPPPGAGGPPMPPPPPPPPPPPSSGNGPAPPPLPPALVPAGGLAPGGGRGALLDQIRQGIQLNKTPGAPESSALQPPPQSSEGLVGALMHVMQKRSRAIHSSDEGEDQAGDEDEDDEWDD

SEQ ID NO: 11 - DNA, 1227 bp, Homo sapiens
  misc_feature (1)..(1227): 98N-WASP
tttgtatataatagtcctagaggatattttcatacctttgctggagatacttgtcaagttgctcttaattttgccaatgaagaagaagcaaaaaaatttcgaaaagcagttacagaccttttgggccgtcgacaaaggaaatctgagaaaagacgagatcccccaaatggtcctaatctacccatggctacagttgatataaaaaatccagaaatcacaacaaatagattttatggtccacaagtcaacaacatctcccataccaaagaaaagaagaagggaaaagctaaaaagaagagattaaccaagggagatataggaacaccaagcaatttccagcacattggacatgttggttgggatccaaatacaggctctgatctgaataatttggatccagaattgaagaatctttttgatatgtgtggaatcttagaggcacaacttaaagaaagagaaacattaaaagttatatatgactttattgaaaaaacaggaggtgttgaagctgttaaaaatgaactgcggaggcaagcaccaccacctccaccaccatcaaggggagggccacctcctcctcctccccctccacatagctcgggtcctcctcctcctcctgctaggggaagaggcgctcctcccccaccaccttcaagagctcccacagctgcacctccaccaccgcctccttccaggccaagtgtagaagtccctccaccaccgccaaataggatgtaccctcctccacctccagcccttccctcctcagcaccttcagggcctccaccaccacctccatctgtgttgggggtagggccagtggcaccacccccaccgcctccacctccacctcctcctgggccaccgcccccgcctggcctgccttctgatggggaccatcaggttccaactactgcaggaaacaaagcagctcttttagatcaaattagagagggtgctcagctaaaaaaagtggagcagaacagtcggccagtgtcctgctctggacgagatgcactgttagaccagatacgacagggtatccaactaaaatctgtggctgatggccaagagtctacaccaccaacacctgcacccacttcaggaattgtgggtgcattaatggaagtgatgcagaaaaggagcaaagccattcattcttcagatgaagatgaagatgaagatgatgaagaagattttgaggatgatgatgagtgggaagactag

SEQ ID NO: 12 - protein, 408 aa, Homo sapiens
  misc_feature (1)..(408): 98N-WASP
FVYNSPRGYFHTFAGDTCQVALNFANEEEAKKFRKAVTDLLGRRQRKSEKRRDPPNGPNLPMATVDIKNPEITTNRFYGPQVNNISHTKEKKKGKAKKKRLTKADIGTPSNFQHIGHVGWDPNTGFDLNNLDPELKNLFDMCGISEAQLKDRETSKVIYDFIEKTGGVEAVKNELRRQAPPPPPPSRGGPPPPPPPPHNSGPPPPPARGRGAPPPPPSRAPTAAPPPPPPSRPSVAVPPPPPNRMYPPPPPALPSSAPSGPPPPPPSVLGVGPVAPPPPPPPPPPPGPPPPPGLPSDGDHQVPTTAGNKAALLDQIREGAQLKKVEQNSRPVSCSGRDALLDQIRQGIQLKSVADGQESTPPTPAPTSGIVGALMEVMQKRSKAIHSSDEDEDEDDEEDFEDDDEWED

SEQ ID NO: 13 - DNA, 2148 bp, Artificial
  Nucleotide sequence encoding Myc-WASP-TAP fusion protein
atgggagagcagaaactgatctctgaagaagacctgaacgatccatcacactggcggccgcagatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggccctcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggccctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgacgagcggccgctcgagaccatggaaaagagaagatggaaaaagaatttcatagccgtctcagcagccaaccgctttaagaaaatctcatcctccggggcacttgattatgatattccaactactgctagcgagaatttgtattttcagggtgagctcaaaaccgcggctcttgcgcaacacgatgaagccgtggacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatggtgctcaggcgccgaaagtagacgcgaattccgcggggaagtcaaccggatccatctag

SEQ ID NO: 14 - protein, 715 aa, Artificial
  Myc-WASP-TAP fusion protein
MGEQKLISEEDLNDPSHWRPQMSGGPMGGRPGGRGAPAVQQNIPSTLLQDHENQRLFEMLGRKCLTLATAVVQLYLALPPGAEHWTKEHCGAVCFVKDNPQKSYFIRLYGLQAGRLLWEQELYSQLVYSTPTPFFHTFAGDDCQAGLNFADEDEAQAFRALVQEKIQKRNQRQSGDRRQLPPPPTPANEERRGGLPPLPLHPGGDQGGPPVGPLSLGLATVDIQNPDITSSRYRGLPAPGPSPADKKRSGKKKISKADIGAPSGFKHVSHVGWDPQNGFDVNNLDPDLRSLFSRAGISEAQLTDAETSKLIYDFIEDQGGLEAVRQEMRRQEPLPPPPPPSRGGNQLPRPPIVGGNKGRSGPLPPVPLGIAPPPPTPRGPPPPGRGGPPPPPPPATGRSGPLPPPPPGAGGPPMPPPPPPPPPPPSSGNGPAPPPLPPALVPAGGLAPGGGRGALLDQIRQGIQLNKTPGAPESSALQPPPQSSEGLVGALMHVMQKRSRAIHSSDEGEDQAGDEDEDDEWDDERPLETMEKRRWKKNFIAVSAANRFKKISSSGALDYDIPTTASENLYFQGELKTAALAQHDEAVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNGAQAPKVDANSAGKSTGSI

SEQ ID NO: 15 - DNA, 2151 bp, Artificial
  Nucleotide sequence encoding Myc-N-WASP-TAP fusion protein
atgggagagcagaaactgatctctgaagaagacctgaacgatccatcacactggcggccgctcgagatgagctccgtccagcagcagccgccgccgccgcggagggtcaccaacgtggggtccctgttgctcaccccgcaggagaacgagtccctcttcactttcctcggcaagaaatgtgtgactatgtcttcagcagtggtgcagttatatgcagcagatcggaactgtatgtggtcaaagaagtgcagtggtgttgcttgtcttgttaaggacaatccacagagatctcattttttaagaatatttgacattaaggatgggaaactattgtgggaacaagagctatacaataactttgtatataatagtcctagaggatattttcatacctttgctggagatacttgtcaagttgctcttaattttgccaatgaagaagaagcaaaaaaatttcgaaaagcagttacagaccttttgggccgtcgacaaaggaaatctgagaaaagacgagatcccccaaatggtcctaatctacccatggctacagttgatataaaaaatccagaaatcacaacaaatagattttatggtccacaagtcaacaacatctcccataccaaagaaaagaagaagggaaaagctaaaaagaagagattaaccaagggagatataggaacaccaagcaatttccagcacattggacatgttggttgggatccaaatacaggctctgatctgaataatttggatccagaattgaagaatctttttgatatgtgtggaatcttagaggcacaacttaaagaaagagaaacattaaaagttatatatgactttattgaaaaaacaggaggtgttgaagctgttaaaaatgaactgcggaggcaagcaccaccacctccaccaccatcaaggggagggccacctcctcctcctccccctccacatagctcgggtcctcctcctcctcctgctaggggaagaggcgctcctcccccaccaccttcaagagctcccacagctgcacctccaccaccgcctccttccaggccaagtgtagaagtccctccaccaccgccaaataggatgtaccctcctccacctccagcccttccctcctcagcaccttcagggcctccaccaccacctccatctgtgttgggggtagggccagtggcaccacccccaccgcctccacctccacctcctcctgggccaccgcccccgcctggcctgccttctgatggggaccatcaggttccaactactgcaggaaacaaagcagctcttttagatcaaattagagagggtgctcagctaaaaaaagtggagcagaacagtcggccagtgtcctgctctggacgagatgcactgttagaccagatacgacagggtatccaactaaaatctgtggctgatggccaagagtctacaccaccaacacctgcacccacttcaggaattgtgggtgcattaatggaagtgatgcagaaaaggagcaaagccattcattcttcagatgaagatgaagatgaagatgatgaagaagattttgaggatgatgatgagtgggaagacctcgagaccatggaaaagagaagatggaaaaagaatttcatagccgtctcagcagccaaccgctttaagaaaatctcatcctccggggcacttgattatgatattccaactactgctagcgagaatttgtattttcagggtgagctcaaaaccgcggctcttgcgcaacacgatgaagccgtggacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatggtgctcaggcgccgaaagtagacgcgaattccgcggggaagtcaaccggatccatctag

SEQ ID NO: 16 - protein, 716 aa, Artificial
  Myc-N-WASP-TAP fusion protein
MGEQKLISEEDLNDPSHWRPLEMSSVQQQPPPPRRVTNVGSLLLTPQENESLFTFLGKKCVTMSSAVVQLYAADRNCMWSKKCSGVACLVKDNPQRSHFLRIFDIKDGKLLWEQELYNNFVYNSPRGYFHTFAGDTCQVALNFANEEEAKKFRKAVTDLLGRRQRKSEKRRDPPNGPNLPMATVDIKNPEITTNRFYGPQVNNISHTKEKKKGKAKKKRLTKGDIGTPSNFQHIGHVGWDPNTGSDLNNLDPELKNLFDMCGILEAQLKERETLKVIYDFIEKTGGVEAVKNELRRQAPPPPPPSRGGPPPPPPPPHSSGPPPPPARGRGAPPPPPSRAPTAAPPPPPPSRPSVEVPPPPPNRMYPPPPPALPSSAPSGPPPPPPSVLGVGPVAPPPPPPPPPPPGPPPPPGLPSDGDHQVPTTAGNKAALLDQIREGAQLKKVEQNSRPVSCSGRDALLDQIRQGIQLKSVADGQESTPPTPAPTSGIVGALMEVMQKRSKAIHSSDEDEDEDDEEDFEDDDEWEDLETMEKRRWKKNFIAVSAANRFKKISSSGALDYDIPTTASENLYFQGELKTAALAQHDEAVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNGAQAPKVDANSAGKSTGSI

SEQ ID NO: 17 - DNA, 1950 bp, Artificial
  Nucleotide sequence encoding GST-105WASP fusion protein
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtccatggtcgaatcaaacaagtttgtacaaaaaagcaggctccgcggccgcccccttcaccgaaaacctgtattttcagggccttgtctactccacccccacccccttcttccacaccttcgctggagatgactgccaagcggggctgaactttgcagacgaggacgaggcccaggccttccgggccctcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggccctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactag

SEQ ID NO: 18 - protein, 649 aa, Artificial
  GST-105WASP fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRPWSNQTSLYKKAGSAAAPFTENLYFQGLVYSTPTPFFHTFAGDDCQAGLNFADEDEAQAFRALVQEKIQKRNQRQSGDRRQLPPPPTPANEERRGGLPPLPLHPGGDQGGPPVGPLSLGLATVDIQNPDITSSRYRGLPAPGPSPADKKRSGKKKISKADIGAPSGFKHVSHVGWDPQNGFDVNNLDPDLRSLFSRAGISEAQLTDAETSKLIYDFIEDQGGLEAVRQEMRRQEPLPPPPPPSRGGNQLPRPPIVGGNKGRSGPLPPVPLGIAPPPPTPRGPPPPGRGGPPPPPPPATGRSGPLPPPPPGAGGPPMPPPPPPPPPPPSSGNGPAPPPLPPALVPAGGLAPGGGRGALLDQIRQGIQLNKTPGAPESSALQPPPQSSEGLVGALMHVMQKRSRAIHSSDEGEDQAGDEDEDDEWDD

SEQ ID NO: 19 - DNA, 1827 bp, Artificial
  Nucleotide encoding Myc-105WASP-TAP
atgggagagcagaaactgatctctgaagaagacctgaacgatccatcacactggcggccgctcgagcttgtctactccacccccacccccttcttccacaccttcgctggagatgactgccaagcggggctgaactttgcagacgaggacgaggcccaggccttccgggccctcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggccctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgacctcgagaccatggaaaagagaagatggaaaaagaatttcatagccgtctcagcagccaaccgctttaagaaaatctcatcctccggggcacttgattatgatattccaactactgctagcgagaatttgtattttcagggtgagctcaaaaccgcggctcttgcgcaacacgatgaagccgtggacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatggtgctcaggcgccgaaagtagacgcgaattccgcggggaagtcaaccggatccatc

SEQ ID NO: 20 - protein, 609 aa, Artificial
  Myc-105WASP-TAP
MGEQKLISEEDLNDPSHWRPLELVYSTPTPFFHTFAGDDCQAGLNFADEDEAQAFRALVQEKIQKRNQRQSGDRRQLPPPPTPANEERRGGLPPLPLHPGGDQGGPPVGPLSLGLATVDIQNPDITSSRYRGLPAPGPSPADKKRSGKKKISKADIGAPSGFKHVSHVGWDPQNGFDVNNLDPDLRSLFSRAGISEAQLTDAETSKLIYDFIEDQGGLEAVRQEMRRQEPLPPPPPPSRGGNQLPRPPIVGGNKGRSGPLPPVPLGIAPPPPTPRGPPPPGRGGPPPPPPPATGRSGPLPPPPPGAGGPPMPPPPPPPPPPPSSGNGPAPPPLPPALVPAGGLAPGGGRGALLDQIRQGIQLNKTPGAPESSALQPPPQSSEGLVGALMHVMQKRSRAIHSSDEGEDQAGDEDEDDEWDDLETMEKRRWKKNFIAVSAANRFKKISSSGALDYDIPTTASENLYFQGELKTAALAQHDEAVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNGAQAPKVDANSAGKSTGSI

SEQ ID NO: 21 - DNA, 1962 bp, Artificial
  Nucleotide sequence encoding GST-tev-98N-WASP fusion protein
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtccatggtcgaatcaaacaagtttgtacaaaaaagcaggcttcgaaaacctgtattttcagggctttgtatataatagtcctagaggatattttcatacctttgctggagatacttgtcaagttgctcttaattttgccaatgaagaagaagcaaaaaaatttcgaaaagcagttacagaccttttgggccgtcgacaaaggaaatctgagaaaagacgagatcccccaaatggtcctaatctacccatggctacagttgatataaaaaatccagaaatcacaacaaatagattttatggtccacaagtcaacaacatctcccataccaaagaaaagaagaagggaaaagctaaaaagaagagattaaccaaggcagatataggaacaccaagcaatttccagcacattggacatgttggttgggatccaaatacaggctttgatctgaataatttggatccagaattgaagaatcttttcgatatgtgtggaatctcagaggcacaacttaaagacagagaaacatcaaaagttatatatgactttattgaaaaaacaggaggtgttgaagctgttaaaaatgaactgcggaggcaagcaccaccacctccaccaccatcaaggggagggccacctcctcctcctccccctccacacaactcaggtcctcctcctcctcctgctaggggaagaggcgctcctcccccaccaccttcaagagctcccacagctgcacctccaccaccgcctccttccaggccaagtgtagcagtccctccaccaccgccaaataggatgtaccctcctccacctccagcccttccctcctcagcaccttcagggcctccaccaccacctccatctgtgttgggggtagggccagtggcaccacccccaccgcctccacctccacctcctcctgggccaccgcccccgcctggcctgccttctgatggggaccatcaggttccaactactgcaggaaacaaagcagctcttttagatcaaattagagagggtgctcagctaaaaaaagtggagcagaacagtcggccagtgtcctgctctggacgagatgcactgttagaccagatacgacagggtatccaactaaaatctgtggctgatggccaagagtctacaccaccaacacctgcacccacttcaggaattgtgggtgcattaatggaagtgatgcagaaaaggagcaaagccattcattcttcagatgaagatgaagatgaagatgatgaagaagattttgaggatgatgatgagtgggaagactag

SEQ ID NO: 22 - protein, 649 aa, Artificial; identical to SEQ ID NO: 18
  GST-tev-98N-WASP fusion protein

SEQ ID NO: 23 - DNA, 1914 bp, Artificial
  Nucleotide encoding Myc-98N-WASP-TAP
atgggagagcagaaactgatctctgaagaagacctgaacgatatcacaagtttgtacaaaaaagcaggcttctttgtatataatagtcctagaggatattttcatacctttgctggagatacttgtcaagttgctcttaattttgccaatgaagaagaagcaaaaaaatttcgaaaagcagttacagaccttttgggccgtcgacaaaggaaatctgagaaaagacgagatcccccaaatggtcctaatctacccatggctacagttgatataaaaaatccagaaatcacaacaaatagattttatggtccacaagtcaacaacatctcccataccaaagaaaagaagaagggaaaagctaaaaagaagagattaaccaaggcagatataggaacaccaagcaatttccagcacattggacatgttggttgggatccaaatacaggctttgatctgaataatttggatccagaattgaagaatcttttcgatatgtgtggaatctcagaggcacaacttaaagacagagaaacatcaaaagttatatatgactttattgaaaaaacaggaggtgttgaagctgttaaaaatgaactgcggaggcaagcaccaccacctccaccaccatcaaggggagggccacctcctcctcctccccctccacacaactcaggtcctcctcctcctcctgctaggggaagaggcgctcctcccccaccaccttcaagagctcccacagctgcacctccaccaccgcctccttccaggccaagtgtagcagtccctccaccaccgccaaataggatgtaccctcctccacctccagcccttccctcctcagcaccttcagggcctccaccaccacctccatctgtgttgggggtagggccagtggcaccacccccaccgcctccacctccacctcctcctgggccaccgcccccgcctggcctgccttctgatggggaccatcaggttccaactactgcaggaaacaaagcagctcttttagatcaaattagagagggtgctcagctaaaaaaagtggagcagaacagtcggccagtgtcctgctctggacgagatgcactgttagaccagatacgacagggtatccaactaaaatctgtggctgatggccaagagtctacaccaccaacacctgcacccacttcaggaattgtgggtgcattaatggaagtgatgcagaaaaggagcaaagccattcattcttcagatgaagatgaagatgaagatgatgaagaagattttgaggatgatgatgagtgggaagacgacccagctttcttgtacaaagtggttgatatcccatcacactggcggccgctcgagaccatggaaaagagaagatggaaaaagaatttcatagccgtctcagcagccaaccgctttaagaaaatctcatcctccggggcacttgattatgatattccaactactgctagcgagaatttgtattttcagggtgagctcaaaaccgcggctcttgcgcaacacgatgaagccgtggacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatggtgctcaggcgccgaaagtagacgcgaattccgcggggaagtcaaccggatccatc

SEQ ID NO: 24 - protein, 638 aa, Artificial
  Myc-98N-WASP-TAP
MGEQKLISEEDLNDITSLYKKAGFFVYNSPRGYFHTFAGDTCQVALNFANEEEAKKFRKAVTDLLGRRQRKSEKRRDPPNGPNLPMATVDIKNPEITTNRFYGPQVNNISHTKEKKKGKAKKKRLTKADIGTPSNFQHIGHVGWDPNTGFDLNNLDPELKNLFDMCGISEAQLKDRETSKVIYDFIEKTGGVEAVKNELRRQAPPPPPPSRGGPPPPPPPPHNSGPPPPPARGRGAPPPPPSRAPTAAPPPPPPSRPSVAVPPPPPNRMYPPPPPALPSSAPSGPPPPPPSVLGVGPVAPPPPPPPPPPPGPPPPPGLPSDGDHQVPTTAGNKAALLDQIREGAQLKKVEQNSRPVSCSGRDALLDQIRQGIQLKSVADGQESTPPTPAPTSGIVGALMEVMQKRSKAIHSSDEDEDEDDEEDFEDDDEWEDDPAFLYKVVDIPSHWRPLETMEKRRWKKNFIAVSAANRFKKISSSGALDYDIPTTASENLYFQGELKTAALAQHDEAVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNGAQAPKVDANSAGKSTGSI

SEQ ID NO: 25 - DNA, 1308 bp, Artificial
  Nucleotide sequence encoding GST-Cdc42 fusion protein
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtccatggtcgaatcaaacaagtttgtacaaaaaagcaggcttcgaaaacctgtattttcagggccagacaattaagtgtgttgttgtgggcgatgttgctgttggtaaaacatgtctcctgatatcctacacaacaaacaaatttccatcggaatatgtaccgactgtttttgacaactatgcagtcacagttatgattggtggagaaccatatactcttggactttttgatactgcagggcaagaggattatgacagattacgaccgctgagttatccacaaacagatgtatttctagtctgtttttcagtggtctctccatcttcatttgaaaacgtgaaagaaaagtgggtgcctgagataactcaccactgtccaaagactcctttcttgcttgttgggactcaaattgatctcagagatgacccctctactattgagaaacttgccaagaacaaacagaagcctatcactccagagactgctgaaaagctggcccgtgacctgaaggctgtcaagtatgtggagtgttctgcacttacacagagaggtctgaagaatgtgtttgatgaggctatcctagctgccctcgagcctccggaaactcaacccaaaaggaagtgctgtatattctag

SEQ ID NO: 26 - protein, 435 aa, Artificial
  GST-Cdc42 fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRPWSNQTSLYKKAGFENLYFQGQTIKCVVVGDVAVGKTCLLISYTTNKFPSEYVPTVFDNYAVTVMIGGEPYTLGLFDTAGQEDYDRLRPLSYPQTDVFLVCFSVVSPSSFENVKEKWVPEITHHCPKTPFLLVGTQIDLRDDPSTIEKLAKNKQKPITPETAEKLARDLKAVKYVECSALTQRGLKNVFDEAILAALEPPETQPKRKCCIF

SEQ ID NO: 27 - DNA, 1314 bp, Artificial
  Nucleotide sequence encoding GST-tev-RhoC fusion protein
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtccatggtcgaatcaaacaagtttgtacaaaaaagcaggcttcgaaaacctgtattttcagggcgctgcaatccgaaagaagctggtgatcgttggggatgttgcctgtgggaagacctgcctcctcatcgtcttcagcaaggatcagtttccggaggtctacgtccctactgtctttgagaactatattgcggacattgaggtggacggcaagcaggtggagctggctctgtgggacacagcagggcaggaagactatgatcgactgcggcctctctcctacccggacactgatgtcatcctcatgtgcttctccatcgacagccctgacagcctggaaaacattcctgagaagtggaccccagaggtgaagcacttctgccccaacgtgcccatcatcctggtggggaataagaaggacctgaggcaagacgagcacaccaggagagagctggccaagatgaagcaggagcccgttcggtctgaggaaggccgggacatggcgaaccggatcagtgcctttggctaccttgagtgctcagccaagaccaaggagggagtgcgggaggtgtttgagatggccactcgggctggcctccaggtccgcaagaacaagcgtcggaggggctgtcccattctctag

SEQ ID NO: 28 - protein, 437 aa, Artificial
  GST-tev-RhoC fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRPWSNQTSLYKKAGFENLYFQGAAIRKKLVIVGDVACGKTCLLIVFSKDQFPEVYVPTVFENYIADIEVDGKQVELALWDTAGQEDYDRLRPLSYPDTDVILMCFSIDSPDSLENIPEKWTPEVKHFCPNVPIILVGNKKDLRQDEHTRRELAKMKQEPVRSEEGRDMANRISAFGYLECSAKTKEGVREVFEMATRAGLQVRKNKRRRGCPIL

SEQ ID NO: 29 - DNA, 1314 bp, Artificial
  Nucleotide sequence encoding GST-tev-RhoA fusion protein
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtccatggtcgaatcaaacaagtttgtacaaaaaagcaggcttcgaaaacctgtattttcagggcgctgccatccggaagaaactggtgattgttggtgatgtagcctgtggaaagacatgcttgctcatagtcttcagcaaggaccagttcccagaggtgtatgtgcccacagtgtttgagaactatgtggcagatatcgaggtggatggaaagcaggtagagttggctttgtgggacacagctgggcaggaagattatgatcgcctgaggcccctctcctacccagataccgatgttatactgatgtgtttttccatcgacagccctgatagtttagaaaacatcccagaaaagtggaccccagaagtcaagcatttctgtcccaacgtgcccatcatcctggttgggaataagaaggatcttcggaatgatgagcacacaaggcgggagctagccaagatgaagcaggagccggtgaaacctgaagaaggcagagatatggcaaacaggattggcgcttttgggtacatggagtgttcagcaaagaccaaagatggagtgagagaggtttttgaaatggctacgagagctgctctgcaagctagacgtgggaagaaaaaatctggttgccttgtcttgtga

SEQ ID NO: 30 - protein, 437 aa, Artificial
  GST-tev-RhoA fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRPWSNQTSLYKKAGFENLYFQGAAIRKKLVIVGDVACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 31 - DNA, 1311 bp, Artificial
  Nucleotide sequence encoding GST-tev-Rac1 fusion protein
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtccatggtcgaatcaaacaagtttgtacaaaaaagcaggcttcgaaaacctgtattttcagggccaggccatcaagtgtgtggtggtgggagacgtagctgtaggtaaaacttgcctactgatcagttacacaaccaatgcatttcctggagaatatatccctactgtctttgacaattattctgccaatgttatggtagatggaaaaccggtgaatctgggcttatgggatacagctggacaagaagattatgacagattacgccccctatcctatccgcaaacagatgtgttcttaatttgcttttcccttgtgagtcctgcatcatttgaaaatgtccgtgcaaagtggtatcctgaggtgcggcaccactgtcccaacactcccatcatcctagtgggaactaaacttgatcttagggatgataaagacacgatcgagaaactgaaggagaagaagctgactcccatcacctatccgcagggtctagccatggctaaggagattggtgctgtaaaatacctggagtgctcggcgctcacacagcgaggcctcaagacagtgtttgacgaagcgatccgagcagtcctctgcccgcctcccgtgaagaagaggaagagaaaatgcctgctgttgtaa

SEQ ID NO: 32 - protein, 436 aa, Artificial
  GST-tev-Rac1 fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRPWSNQTSLYKKAGFENLYFQGQAIKCVVVGDVAVGKTCLLISYTTNAFPGEYIPTVFDNYSANVMVDGKPVNLGLWDTAGQEDYDRLRPLSYPQTDVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 33 - DNA, 1845 bp, Artificial
  Nucleotide sequence encoding GST-tev-Nck1 fusion protein
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtccatggtcgaatcaaacaagtttgtacaaaaaagcaggcttcgcagaagaagtggtggtagtagccaaatttgattatgtggcccaacaagaacaagagttggacatcaagaagaatgagagattatggcttctggatgattctaagtcctggtggcgagttcgaaattccatgaataaaacaggttttgtgccttctaactatgtggaaaggaaaaacagtgctcggaaagcatctattgtgaaaaacctaaaggataccttaggcattggaaaagtgaaaagaaaacctagtgtgccagattctgcatctcctgctgatgatagttttgttgacccaggggaacgtctctatgacctcaacatgcccgcttatgtgaaatttaactacatggctgagagagaggatgaattatcattgataaaggggacaaaggtgatcgtcatggagaaatgcagtgatgggtggtggcgtggtagctacaatggacaagttggatggttcccttcaaactatgtaactgaagaaggtgacagtcctttgggtgaccatgtgggttctctgtcagagaaattagcagcagtcgtcaataacctaaatactgggcaagtgttgcatgtggtacaggctctttacccattcagctcatctaatgatgaagaacttaatttcgagaaaggagatgtaatggatgttattgaaaaacctgaaaatgacccagagtggtggaaatgcaggaagatcaatggtatggttggtctagtaccaaaaaactatgttaccgttatgcagaataatccattaacttcaggtttggaaccatcacctccacagtgtgattacattaggccttcactcactggaaagtttgctggcaatccttggtattatggcaaagtcaccaggcatcaagcagaaatggcattaaatgaaagaggacatgaaggggatttcctcattcgtgatagtgaatcttcgccaaatgatttctcagtatcactaaaagcacaagggaaaaacaagcattttaaagtccaactaaaagagactgtctactgcattgggcagcgtaaattcagcaccatggaagaacttgtagaacattacaaaaaggcaccaatttttacaagtgaacaaggagaaaaattatatcttgtcaagcatttatcatga

SEQ ID NO: 34 - protein, 614 aa, Artificial
  GST-tev-Nck1 fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRPWSNQTSLYKKAGFAEEVVVVAKFDYVAQQEQELDIKKNERLWLLDDSKSWWRVRNSMNKTGFVPSNYVERKNSARKASIVKNLKDTLGIGKVKRKPSVPDSASPADDSFVDPGERLYDLNMPAYVKFNYMAEREDELSLIKGTKVIVMEKCSDGWWRGSYNGQVGWFPSNYVTEEGDSPLGDHVGSLSEKLAAVVNNLNTGQVLHVVQALYPFSSSNDEELNFEKGDVMDVIEKPENDPEWWKCRKINGMVGLVPKNYVTVMQNNPLTSGLEPSPPQCDYIRPSLTGKFAGNPWYYGKVTRHQAEMALNERGHEGDFLIRDSESSPNDFSVSLKAQGKNKHFKVQLKETVYCIGQRKFSTMEELVEHYKKAPIFTSEQGEKLYLVKHLS

SEQ ID NO: 35 - DNA, 561 bp, Artificial
  Nucleotide sequence encoding TAP tag (i.e., CBP, tev cleavage site and Prot A)
atggaaaagagaagatggaaaaagaatttcatagccgtctcagcagccaaccgctttaagaaaatctcatcctccggggcacttgattatgatattccaactactgctagcgagaatttgtattttcagggtgagctcaaaaccgcggctcttgcgcaacacgatgaagccgtggacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatggtgctcaggcgccgaaagtagacgcgaattccgcggggaagtcaaccggatccatctag

SEQ ID NO: 36 - protein, 186 aa, Artificial
  TAP tag (i.e., CBP, tev cleavage site and Prot A)
MEKRRWKKNFIAVSAANRFKKISSSGALDYDIPTTASENLYFQGELKTAALAQHDEAVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNGAQAPKVDANSAGKSTGSI

SEQ ID NO: 37 - DNA, 1143 bp, Homo sapiens
  misc_feature (1)..(1143): NCK2
atgacagaagaagttattgtgatagccaagtgggactacaccgcccagcaggaccaggagctggacatcaagaagaacgagcggctgtggttgctggacgactccaagacgtggtggcgggtgaggaacgcggccaacaggacgggctatgtaccgtccaactacgtggagcggaagaacagcctgaagaagggctccctcgtgaagaacctgaaggacacactaggcctcggcaagacgcgcaggaagaccagcgcgcgggatgcgtcccccacgcccagcacggacgccgagtaccccgccaatggcagcggcgccgaccgcatctacgacctcaacatcccggccttcgtcaagttcgcctatgtggccgagcgggaggatgagttgtccctggtgaaggggtcgcgcgtcaccgtcatggagaagtgcagcgacggttggtggcggggcagctacaacgggcagatcggctggttcccctccaactacgtcttggaggaggtggacgaggcggctgcggagtccccaagcttcctgagcctgcgcaagggcgcctcgctgagcaatggccagggctcccgcgtgctgcatgtggtccagacgctgtaccccttcagctcagtcaccgaggaggagctcaacttcgagaagggggagaccatggaggtgattgagaagccggagaacgaccccgagtggtggaaatgcaaaaatgcccggggccaggtgggcctcgtccccaaaaactacgtggtggtcctcagtgacgggcctgccctgcaccctgcgcacgccccacagataagctacaccgggccctcgtccagcgggcgcttcgcgggcagagagtggtactacgggaacgtgacgcggcaccaggccgagtgcgccctcaacgagcggggcgtggagggcgacttcctcattagggacagcgagtcctcgcccagcgacttctccgtgtcccttaaagcgtcagggaagaacaaacacttcaaggtgcagctcgtggacaatgtctactgcattgggcagcggcgcttccacaccatggacgagctggtggaacactacaaaaaggcgcccatcttcaccagcgagcacggggagaagctctacctcgtcagggccctgcagtga

SEQ ID NO: 38 - protein, 380 aa, Homo sapiens
  misc_feature (1)..(380): NCK2
MTEEVIVIAKWDYTAQQDQELDIKKNERLWLLDDSKTWWRVRNAANRTGYVPSNYVERKNSLKKGSLVKNLKDTLGLGKTRRKTSARDASPTPSTDAEYPANGSGADRIYDLNIPAFVKFAYVAEREDELSLVKGSRVTVMEKCSDGWWRGSYNGQIGWFPSNYVLEEVDEAAAESPSFLSLRKGASLSNGQGSRVLHVVQTLYPFSSVTEEELNFEKGETMEVIEKPENDPEWWKCKNARGQVGLVPKNYVVVLSDGPALHPAHAPQISYTGPSSSGRFAGREWYYGNVTRHQAECALNERGVEGDFLIRDSESSPSDFSVSLKASGKNKHFKVQLVDNVYCIGQRRFHTMDELVEHYKKAPIFTSEHGEKLYLVRALQ

SEQ ID NO: 39 - DNA, 1854 bp, Homo sapiens
  misc_feature (1)..(1854): GST_NCK2
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtccatggtcgaatcaaacaagtttgtacaaaaaagcaggcttcacagaagaagttattgtgatagccaagtgggactacaccgcccagcaggaccaggagctggacatcaagaagaacgagcggctgtggttgctggacgactccaagacgtggtggcgggtgaggaacgcggccaacaggacgggctatgtaccgtccaactacgtggagcggaagaacagcctgaagaagggctccctcgtgaagaacctgaaggacacactaggcctcggcaagacgcgcaggaagaccagcgcgcgggatgcgtcccccacgcccagcacggacgccgagtaccccgccaatggcagcggcgccgaccgcatctacgacctcaacatcccggccttcgtcaagttcgcctatgtggccgagcgggaggatgagttgtccctggtgaaggggtcgcgcgtcaccgtcatggagaagtgcagcgacggttggtggcggggcagctacaacgggcagatcggctggttcccctccaactacgtcttggaggaggtggacgaggcggctgcggagtccccaagcttcctgagcctgcgcaagggcgcctcgctgagcaatggccagggctcccgcgtgctgcatgtggtccagacgctgtaccccttcagctcagtcaccgaggaggagctcaacttcgagaagggggagaccatggaggtgattgagaagccggagaacgaccccgagtggtggaaatgcaaaaatgcccggggccaggtgggcctcgtccccaaaaactacgtggtggtcctcagtgacgggcctgccctgcaccctgcgcacgccccacagataagctacaccgggccctcgtccagcgggcgcttcgcgggcagagagtggtactacgggaacgtgacgcggcaccaggccgagtgcgccctcaacgagcggggcgtggagggcgacttcctcattagggacagcgagtcctcgcccagcgacttctccgtgtcccttaaagcgtcagggaagaacaaacacttcaaggtgcagctcgtggacaatgtctactgcattgggcagcggcgcttccacaccatggacgagctggtggaacactacaaaaaggcgcccatcttcaccagcgagcacggggagaagctctacctcgtcagggccctgcagtga

SEQ ID NO: 40 - protein, 617 aa, Homo sapiens
  misc_feature (1)..(617): GST_NCK2
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRPWSNQTSLYKKAGFTEEVIVIAKWDYTAQQDQELDIKKNERLWLLDDSKTWWRVRNAANRTGYVPSNYVERKNSLKKGSLVKNLKDTLGLGKTRRKTSARDASPTPSTDAEYPANGSGADRIYDLNIPAFVKFAYVAEREDELSLVKGSRVTVMEKCSDGWWRGSYNGQIGWFPSNYVLEEVDEAAAESPSFLSLRKGASLSNGQGSRVLHVVQTLYPFSSVTEEELNFEKGETMEVIEKPENDPEWWKCKNARGQVGLVPKNYVVVLSDGPALHPAHAPQISYTGPSSSGRFAGREWYYGNVTRHQAECALNERGVEGDFLIRDSESSPSDFSVSLKASGKNKHFKVQLVDNVYCIGQRRFHTMDELVEHYKKAPIFTSEHGEKLYLVRALQ

SEQ ID NO: 41 - DNA, 77 bp, Artificial
  WASP forward primer
ggggacaagtttgtacaaaaaagcaggcttcgaaaacctgtattttcagggcgggggtcggggagcgcttttggatc

SEQ ID NO: 42 - DNA, 57 bp, Artificial
  WASP reverse primer
ggggaccactttgtacaagaaagctgggtcctagtcatcccattcatcatcttcatc

SEQ ID NO: 43 - DNA, 49 bp, Artificial
  WASP forward primer
caccgaaaacctgtattttcagggccttgtctactccacccccaccccc

SEQ ID NO: 44 - DNA, 24 bp, Artificial
  WASP reverse primer
ctagtcatcccattcatcatcttc

SEQ ID NO: 45 - DNA, 55 bp, Artificial
  WASP forward primer
ggggacaagtttgtacaaaaaagcaggcttccttgtctactccacccccaccccc

SEQ ID NO: 46 - DNA, 54 bp, Artificial
  WASP reverse primer
ggggaccactttgtacaagaaagctgggtcgtcatcccattcatcatcttcatc

SEQ ID NO: 47 - DNA, 54 bp, Artificial
  WASP forward primer
ggggacaagtttgtacaaaaaagcaggcttcatgagtgggggcccaatgggagg

SEQ ID NO: 48 - DNA, 70 bp, Artificial
  NWASP forward primer
ggggacaagtttgtacaaaaaagcaggcttcgaaaacctgtattttcagggctctgatggggaccatcag

SEQ ID NO: 49 - DNA, 57 bp, Artificial
  NWASP reverse primer
ggggaccactttgtacaagaaagctgggtcctagtcttcccactcatcatcatcctc

SEQ ID NO: 50 - DNA, 56 bp, Artificial
  pENTR/SD/TOPO_NWASP forward primer
caccgaaaacctgtattttcagggctttgtatataatagtcctagaggatattttc

SEQ ID NO: 51 - DNA, 24 bp, Artificial
  pENTR/SD/TOPO_NWASP reverse primer
ttagtcttcccactcatcatcatc

SEQ ID NO: 52 - DNA, 75 bp, Artificial
  pENTR/SD/TOPO_tev_98FNWASP forward primer
ggggacaagtttgtacaaaaaagcaggcttcgaaaacctgtattttcagggctttgtatataatagtcctagagg

SEQ ID NO: 53 - DNA, 54 bp, Artificial
  pENTR/SD/TOPO_tev_98FNWASP reverse primer
ggggaccactttgtacaagaaagctgggtcgtcttcccactcatcatcatcctc

SEQ ID NO: 54 - DNA, 49 bp, Artificial
  NWASP forward primer
caccgaaaacctgtattttcagggcagctccgtccagcagcagccgccg

SEQ ID NO: 55 - DNA, 24 bp, Artificial
  NWASP reverse primer
tcagtcttcccactcatcatcatc

SEQ ID NO: 56 - DNA, 31 bp, Artificial
  pENTR_N-WASP/SD/TOPO forward primer
gccgctcgaggtcttcccactcatcatcatc

SEQ ID NO: 57 - DNA, 29 bp, Artificial
  pENTR_N-WASP/SD/TOPO reverse primer
gccgctcgagatgagctccgtccagcagc

SEQ ID NO: 58 - DNA, 39 bp, Artificial
  pDONR_tev_Cdc42 GTP forward primer
tgtgttgttgtgggcgatgttgctgttggtaaaacatgt

SEQ ID NO: 59 - DNA, 39 bp, Artificial
  pDONR_tev_Cdc42 GTP reverse primer
acatgttttaccaacagcaacatcgcccacaacaacaca

SEQ ID NO: 60 - DNA, 33 bp, Artificial
  pDONR-tev_RhoC forward primer
gtgatcgttggggatgttgcctgtgggaaggac

SEQ ID NO: 61 - DNA, 33 bp, Artificial
  pDONR-tev_RhoC reverse primer
gtccttcccacaggcaacatccccaacgatcac

SEQ ID NO: 62 - DNA, 76 bp, Artificial
  RhoA GTP forward primer
ggggacaagtttgtacaaaaaagcaggcttcgaaaacctgtattttcagggcgctgccatccggaagaaactggtg

SEQ ID NO: 63 - DNA, 58 bp, Artificial
  RhoA GTP reverse primer
ggggaccactttgtacaagaaagctgggtcctacaagacaaggcaaccacattttttc

SEQ ID NO: 64 - DNA, 76 bp, Artificial
  Rac1 GTP forward primer
ggggacaagtttgtacaaaaaaacgggcttcgaaaacctgtattttcagggccaggccatcaagtgtgtggtggtg

SEQ ID NO: 65 - DNA, 58 bp, Artificial
  Rac1 GTP reverse primer
ggggaccactttgtacaagaaagctgggtcctacaacagcaggcattttctcttcctc

SEQ ID NO: 66 - DNA, 77 bp, Artificial
  Nck forward primer
ggggacaagtttgtacaaaaaagcaggcttcgaaaacctgtattttcagggcatggcagaagaagtggtggtagtag

SEQ ID NO: 67 - DNA, 57 bp, Artificial
  Nck reverse primer
ggggaccactttgtacaagaaagctgggtcctatgataaatgcttgacaagatataa

SEQ ID NO: 68 - DNA, 31 bp, Artificial
  NCK2 forward primer
caccatgacagaagaagttattgtgatagcc

SEQ ID NO: 69 - DNA, 27 bp, Artificial
  NCK2 reverse primer
tcactgcagggccctgacgaggtagag

SEQ ID NO: 70 - DNA, 84 bp, Artificial
  WASP reverse primer
ggggaccactttgtacaagaaagctgggtcctagtgatggtgatggtgatggtagtacgagtcatcccattcatcatcttcatc

(19) United States
(12) Patent Application Publication
(10) Pub. No.: US 2006/0024786 A1
(43) Pub. Date: Feb. 2, 2006

(54) WASP and N-WASP constructs and methods of expressing such constructs

(75) Inventors: Nenad Tomasevic, Foster City, CA (US); Alan Russell, San Francisco, CA (US); Manping Wang, Union City, CA (US); Roman Sakowicz, Foster City, CA (US); Christophe Beraud, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Cytokinetics, Inc., South San Francisco, CA (US)

(21) Appl. No.: 11/150,487

(22) Filed: Jun. 10, 2005

Related U.S. Application Data
(60) Provisional application No. 60/578,913, filed on Jun. 10, 2004.

(51) Int. Cl. C07H 21/04; C12P 21/06; C12N 9/16

(52) U.S. Cl. 435/69.1; 435/196; 435/320.1; 435/325; 536/23.2

(57) ABSTRACT

WASP and N-WASP proteins are provided that can be expressed in soluble form, including fusion proteins that contain full-length WASP and N-WASP. The proteins retain at least partial activity of full length WASP or N-WASP and some of the proteins fully recapitulate the activity of full-length WASP and N-WASP. Methods for expressing full-length WASP and N-WASP are also provided.

### CROSS-REFERENCES TO RELATED APPLICATIONS

This application claims the benefit of U.S. Provisional Application No. 60/578,913, filed Jun. 10, 2004, which is incorporated herein by reference in its entirety for all purposes. This application is related to U.S. application Ser. No. ______, filed ______, which claims the benefit of U.S. Provisional Application Nos. 60/578,949, filed Jun. 10, 2004, and 60/673,444, filed Apr. 20, 2005, all of which are incorporated herein by reference in their entirety for all purposes. This application is also related to U.S. application Ser. No. ______, filed ______, which claims the benefit of U.S. Provisional Application No. 60/578,969, filed Jun. 10, 2004, both of which are incorporated herein by reference in their entirety for all purposes.

### BACKGROUND

The actin cytoskeleton and proteins that regulate its formation play a central role in cell movement and polarity, and thus are useful targets for the treatment of inflammatory diseases and for preventing metastatic spread of primary cancers. Polarized cell movement is driven by reorganization of the cortical actin cytoskeleton at the leading edge of moving cells, resulting in the production of a propulsive force (Higgs, H. N. et al. (2001) Annu Rev Biochem 70:649-676; Small, J. V. et al. (2002) Trends Cell Biol 12:112-120). The actin cytoskeleton also plays a role in changes in cell shape and in the internalization of extracellular materials via endocytosis and phagocytosis.

These processes depend upon the rapid and localized assembly and disassembly of actin filaments. New filaments are created by nucleation of monomeric actin (Carson, M. et al. (1986) J. Cell Biol. 103:2707-2714; Chan, A. Y. et al. (1998) J. Cell Sci. 111:199-211), which refers to the initiation of actin polymerization from free actin monomers (globular actin or G-actin) into filamentous actin (F-actin), and is the rate-limiting step in the assembly of actin filaments. The very large kinetic barrier to nucleation indicates that regulation of the nucleation step may be critical to controlling actin polymerization in cells.

The actin nucleation machinery includes at least two key components: the Arp2/3 complex and one or more members from the family of nucleation promoting factors (NPFs). The Arp2/3 complex (or simply Arp2/3) is responsible for nucleating new actin filaments and cross-linking newly formed filaments into Y-branched arrays. In particular, the Arp2/3 complex is positioned at the Y-branch between the filaments and stabilizes the cross-link region. The Arp2/3 complex consists of six subunits in Saccharomyces cerevisiae and seven subunits in Acanthaemoeba castellanii and humans. The two largest subunits (50 and 43 kDa) are actin-related proteins in the Arp3 (also sometimes referred to as ACTR2) and Arp2 (sometimes referred to as ACTR3) families, respectively. The name of the complex is thus named after these two subunits. The other five subunits in the human complex have molecular masses of approximately 41, 34, 21, 20 and 16 kDa, based upon sodium dodecyl sulfate-polyacrylamide gel electrophoresis studies and the subunits from humans are referred to as p41-Arc, p34-Arc, p21-Arc, p20-Arc and p16-Arc, respectively.

Arp2/3 by itself, however, possesses little activity. The complex must be bound by a NPF to become activated. Examples of such NPFs include Wiskott-Aldrich syndrome protein (WASP), a WASP homolog called N-WASP, and a family of proteins called suppressor of cAR (SCAR) (also referred to as the WASP family verprolin homologous (WAVE) proteins). The SCAR/WAVE family includes SCAR1/WAVE1, SCAR2/WAVE2 and SCAR3/WAVE3. See, for example, Welch, M. D. and Mullins, R. D. (2002) Annu. Rev. Cell Dev. Biol. 18:247-288; Higgs, H. N. and Pollard, T. D. (2001) Annu. Rev. Biochem. 70:649-76; Caron, E. (2002) Curr Opin Cell Biol 14:82-87; and Takenawa, T. (2001) J Cell Sci 114:1801-1809, each of which are incorporated herein by reference in their entirety for all purposes. WASP is expressed exclusively in hematopoietic cells, N-WASP and WAVE2 are ubiquitously expressed, and WAVE1 and WAVE3 are expressed exclusively in the neurons.

Once a NPF has bound Arp2/3 to form an activated conformation, the complex serves as a nucleus for polymerization of G-ATP-actin and mimics the barbed end of an actin filament. During the nucleation process, the Arp2/3 complex binds to the side of an existing actin filament. Filament binding in the absence of an activator, or activator interaction in the absence of a pre-existing actin filament, does not result in appreciable Arp2/3 activity. Arp2/3 does not interact with the ends of filaments in any manner except with the filament that itself has nucleated.

NPFs are also regulated. They are activated by the binding of upstream regulatory molecules. Examples of such regulatory proteins involved in the activation of WASP and N-WASP include: 1) the Rho-family GTPase, Cdc42, 2) the acidic lipid, phosphatidylinositol-4,5-bisphosphate (PIP₂), 3) Src family tyrosine kinases, 4) Btk and Itk tyrosine kinases, and 5) syndapin 1. See, e.g., Higgs and Pollard, supra.

Although NPFs such as the WASP/WAVE/SCAR family of proteins exhibit some structural variety and have been shown to interact with a number of different proteins, all members of this family contain a hallmark domain at the C-terminus. It is this domain that mediates WASP/WAVE/SCAR-stimulation of the Arp2/3 complex of proteins and nucleation of actin filaments (see FIG. 1). This C-terminal domain is referred to as the VCA domain (also sometimes referred to as the WWA or simply WA region). The V region (or WH2 region) of the VCA domain is responsible for binding G-actin, whereas the CA region is responsible for binding to and activating the Arp2/3 complex (Miki, H., and Takenawa, T. (1998) Biochem Biophys Res Commun 243:73-8). Other domains shared by members of the WASP/WAVE/SCAR family is a proline rich domain (PolyPro), a basic domain (B) and a N-terminal WASP homology domain (WH1) (see FIG. 1). Upstream regulatory molecules bind to the PolyPro, B and WH1 domains to regulate the activity of the WASP/WAVE/SCAR family of proteins.

WASP and N-WASP are normally present in a folded conformation that prevents exposure of the VCA domain and inactivates the protein (Miki, H. et al. (1998) Nature 391:93-6; and Kim, A. S. et al. (2000) Nature 404:151-8). Activation occurs through two identified routes, which induce unfolding of the protein, exposure of the VCA domain and activation of Arp2/3. The first is through the binding of the Rho family GTPase Cdc42 to the CRIB domain of WASP (Miki, H. et al. (1998) Nature 391:93-6). The second is by binding of the adaptor protein Nck to the proline rich domain (Rohatgi, R. et al. (2001) J. Biol. Chem. 276:26448-52). The N-terminal WH1 domain of WASP also contributes to activity through binding of PIP₂ (Miki, H. et al. (1996) EMBO J. 15:5326-35), which anchors the protein to the cell membrane. The WH1 domain also recruits WASP-interacting protein, WIP (Ramesh, N. et al. (1997) Proc Natl Acad Sci USA 94:14671-6); this protein is involved in both actin polymerization and specialized activation of transcription factors such as NFAT in T cells after recruitment to WASP (Anton, I. M. et al. (2002) Immunity 16:193-204). These concerted functions of WASP in the immune system place it at the center of an essential crossroads between extracellular signaling pathways and coherent cytoskeletal responses. See also Higgs, H. N. and Pollard, T. D. (2001) Annu. Rev. Biochem. 70:649-76.

One line of evidence supporting a role for WASP proteins in mammalian physiology and pathology is derived from the presentation of patients suffering from Wiskott-Aldrich Syndrome. WAS patients are deficient in the eponymous protein, WASP. These patients exhibit a heterogeneous array of symptoms ranging in severity. All WAS patients most commonly suffer from general immunodeficiency, thrombocytopenia, and eczema (Zhu, Q. et al. (1997) Blood 90:2680-2689). T-cells from WAS patients fail to respond to antigen presentation, and WAS monocytes and neutrophils are often found to be defective in chemotaxis responses (Snapper, S. B., and Rosen, F. S. (1999) Annu Rev Immunol 17: 905-929).

Mice expressing a version of WASP lacking the GBD/CRIB domain exhibit a subset of these characteristics (Snapper, S. B. et al. (1998) Immunity 9:81-91). Restriction of the WAS phenotype to haematopoietic cells is consistent with expression of WASP only in haematopoietic tissues. N-WASP is also an essential gene in mice. Targeted disruption of N-WASP causes embryonic lethality (Snapper, S. B. et al. (2001) Nat Cell Biol 3:897-904).

In view of the important role that NPFs such as WASP and N-WASP play in a variety of cellular processes and disease, it would be useful to have methods of rapidly preparing large quantities of these proteins. Attempts to express WASP and N-WASP, however, have experienced several difficulties, including lack of solubility and/or poor activity. This has been particularly true of efforts to express full-length WASP and N-WASP. Expressing a WASP or N-WASP protein that fully recapitulates the activities of the full-length proteins has also proven problematic. There thus remains a need for additional WASP and N-WASP constructs that have the desired activities and methods by which such constructs, including full-length WASP and N-WASP, can be expressed in a soluble and active form.

### BRIEF DESCRIPTION OF THE DRAWINGS

FIG. 1 depicts the major domains in the WASP and WAVE/SCAR family of proteins. All consist of a similar organization of a distinct WASP/SCAR homology domain (WH1/SH1), a basic region (B), and a proline rich region (PolyPro). The actin polymerization machinery consists of one or two verprolin-homology domains (V) a central region, (C) and an acidic domain (A). Interaction between the basic and acidic regions maintains the proteins in an inactive state. WASP and N-WASP also have a GTPase-binding CRIB domain in common.

FIGS. 2A and 2B show the approximate amino acid regions that correspond to the various major domains of WASP and N-WASP, respectively.

FIGS. 3A and 3B show the general structure of some of the WASP proteins that are described herein.

FIG. 4 depicts the extent of purification of full length WASP using certain purification methods described herein.

FIG. 5 is a chart in which fluorescence is plotted as a function of time (seconds). The chart illustrates that full length WASP (FL-WASP) and full length N-WASP (FL N-WASP) alone can only weakly stimulate actin polymerization, but that inclusion of the activators Cdc42 or Nck1 can accelerate actin polymerization 13 times. The significant regulation of FL WASP and FL N-WASP obtained by methods provided herein indicates that the proteins are properly folded.

FIG. 6 is a plot comparing the relative activities of FL-WASP as compared to two truncated forms of WASP: 105-WASP (a version of WASP that lacks the WH1 domain), and the VCA/WA domain. The results shown in this plot demonstrate that FL WASP is approximately 20 times more active than 105-WASP and 70 times more potent than the VCA domain alone.

FIG. 7 is a graph that illustrates the ability of four upstream activators (Cdc42, Nck1, Nck2 and Rac1) to activate FL WASP. The results show that: 1) Nck1 was the most potent activator, 2) Cdc42 in the absence of PIP₂ vesicles fully activate FL WASP and 3) there is a bell shaped dependence between Nck1 and Nck2 and barbed end concentrations.

FIG. 8 is a graph that illustrates the ability of the four upstream activators shown in FIG. 7 to activate N-WASP. The results shown in this figure demonstrate that: 1) Rac 1 activates FL N-WASP, 2) in the absence of PIP₂ that Rac 1 is a more potent N-WASP activator than Cdc42, 3) Nck1 and Nck2 were the only FL N-WASP activators that can stimulate production of maximal concentration of barbed ends, 4) Nck2 is a significantly better activator of N-WASP than WASP, and 5) there is a bell shaped dose dependence curve for Nck1, Nck2 and Rac1.

FIG. 9 is a chart which illustrates the effect that PIP₂ has on the maximal rate of polymerization in the presence of FL WASP and different upstream activators, namely Cdc42, Rac1, Nck1 and Nck2. The chart shows that: 1) PIP₂ had no effect on FL WASP in the absence of small GTPases or Nck and, 2) PIP₂ had a strong inhibitory effect on WASP stimulated actin polymerization in the presence of both small GTPases or Nck.

FIG. 10 is a chart similar to that described in FIG. 9, except that it represents the effect of PIP₂ on the maximal rate of polymerization in the presence of N-WASP and Cdc42, Rac1, Nck1 or Nck2. The chart demonstrates that: 1) PIP₂ had a marked synergistic effect on N-WASP activation by Rac1 or Cdc42, and 2) PIP₂ inhibited Nck stimulated activation of N-WASP.

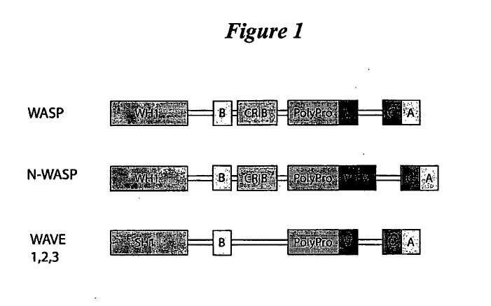

### DETAILED DESCRIPTION

I. Definitions

All technical and scientific terms used herein have the meaning commonly understood by a person skilled in the art to which this invention belongs, including the definitions provided herein. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991).

The terms “nucleic acid,” “polynucleotide,” and “oligonucleotide” are used interchangeably and refer to a deoxyribonucleotide or ribonucleotide polymer in either single-, double, or triple-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties. In general, an analogue of a particular nucleotide has the same base-pairing specificity; i.e., an analogue of A will base-pair with T. The terms additionally encompass nucleic acids containing known nucleotide analogs or modified backbone residues or linkages, that are synthetic, naturally occurring, and non-naturally occurring and that have similar binding properties as the reference nucleic acid. Examples of such analogs include, without limitation, phosphorothioates, phosphoramidates, methyl phosphonates, chiral-methyl phosphonates, 2-O-methyl ribonucleotides, and peptide-nucleic acids (PNAs).

“Polypeptide” and “protein” are used interchangeably herein and include a molecular chain of amino acids linked through peptide bonds. The terms do not refer to a specific length of the product. Thus, “peptides,” “oligopeptides,” and “proteins” are included within the definition of polypeptide. The term also applies to amino acid polymers in which one or more amino acids are chemical analogues of a corresponding naturally-occurring amino acid. In addition, protein fragments, analogs, mutated or variant proteins, fusion proteins and the like are included within the meaning of polypeptide.

A “subsequence” refers to a sequence of nucleotides or amino acids that comprises a part of a longer sequence of nucleotides or amino acids (e.g., a polypeptide), respectively.

A “fusion protein” or “fusion polypeptide” is a molecule in which two or more protein subunits are linked, typically covalently. The subunits can be directly linked or linked via a linking segment. An exemplary fusion protein is one in which a domain from a nucleation promoting factor (e.g., VCA region) is linked to one or more purification tags (e.g., glutathione-S-transferase, His6, an epitope tag, and calmodulin binding protein).

The term “operably linked” or “operatively linked” is used with reference to a juxtaposition of two or more components (e.g., protein domains), in which the components are arranged such that each of the components function normally and allow the possibility that at least one of the components can mediate a function that is exerted upon at least one of the other components. By way of illustration, a transcriptional regulatory sequence (e.g., a promoter) is operably linked to a coding sequence if the transcriptional regulatory sequence controls the level of transcription of the coding sequence in response to the presence or absence of one or more transcriptional regulatory factors. With respect to fusion proteins or polypeptides, the terms can refer to the fact that each of the components performs the same function in the linkage to the other component as it would if it were not so linked. For example, in a fusion protein in which the VCA region of a nucleation promoting factor is fused to a glutathione-S-transferase (GST) tag, these two elements are considered to be operably linked if the VCA region can still bind to and activate Arp2/3 and the GST tag can bind to glutathione (e.g., the glutathione on a glutathione Sepharose matrix).

A “heterologous sequence” or a “heterologous nucleic acid,” as used herein, is one that originates from a source foreign to the particular host cell, or, if from the same source, is modified from its original form. Thus, a heterologous gene in a prokaryotic host cell includes a gene that, although being endogenous to the particular host cell, has been modified. Modification of the heterologous sequence can occur, e.g., by treating the DNA with a restriction enzyme to generate a DNA fragment that is capable of being operably linked to the promoter. Techniques such as site-directed mutagenesis are also useful for modifying a heterologous nucleic acid.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant expression cassette” or simply an “expression cassette” is a nucleic acid construct, generated recombinantly or synthetically, that has control elements that are capable of effecting expression of a structural gene that is operably linked to the control elements in hosts compatible with such sequences. Expression cassettes include at least promoters and optionally, transcription termination signals. Typically, the recombinant expression cassette includes at least a nucleic acid to be transcribed (e.g., a nucleic acid encoding a desired polypeptide) and a promoter. Additional factors necessary or helpful in effecting expression can also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

The terms “identical” or percent “identity,” in the context of two or more nucleic acids or polypeptides, refer to two or more sequences or subsequences that are the same or have a specified percentage of nucleotides or amino acid residues that are the same, when compared and aligned for maximum correspondence, as measured using a sequence comparison algorithm such as those described below for example, or by visual inspection.

The phrase “substantially identical” or “substantial sequence identity,” in the context of two nucleic acids or polypeptides, refers to two or more sequences or subsequences that have at least 70% or 75%, preferably at least 80% or 85%, more preferably at least 90%, 95%, 97%, 99% or higher nucleotide or amino acid residue identity, when compared and aligned for maximum correspondence, as measured using a sequence comparison algorithm such as those described below for example, or by visual inspection. Preferably, the substantial identity exists over a region of the sequences that is at least about 10, 20, 30, 40 or 50 nucleotides or amino acids in length, in some instances over a longer region such as 60, 70 or 80 nucleotides or amino acids, and in other instances over a region of at least about 100, 150, 200, 250, 300, 350 or 400 nucleotides or amino acid residues. And, in still other instances, the sequences are substantially identical over the full length of the sequences being compared, such as the coding region of a nucleotide for example.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection [see generally, Current Protocols in Molecular Biology, (Ausubel, F. M. et al., eds.), John Wiley & Sons, Inc., New York (1987-1999, including supplements such as supplement 46 (April 1999)]. Use of these programs to conduct sequence comparisons are typically conducted using the default parameters specific for each program.

Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra.). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For identifying whether a nucleic acid or polypeptide is within the scope of the invention, the default parameters of the BLAST programs are suitable. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix. The TBLATN program (using protein sequence for nucleotide sequence) uses as defaults a word length (W) of 3, an expectation (E) of 10, and a BLOSUM 62 scoring matrix. (See Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Another indication that two nucleic acid sequences are substantially identical is that the two molecules hybridize to each other under stringent conditions. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA.

A further indication that two nucleic acid sequences or polypeptides are substantially identical is that the polypeptide encoded by the first nucleic acid is immunologically cross reactive with the polypeptide encoded by the second nucleic acid, as described below.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent variations,” which are one species of “conservatively modified variations.” Every polynucleotide sequence described herein which encodes a polypeptide also describes every possible silent variation, except where otherwise noted. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

A polypeptide is typically substantially identical to a second polypeptide, for example, where the two peptides differ only by conservative substitutions. A “conservative substitution,” when describing a protein, refers to a change in the amino acid composition of the protein that does not substantially alter the protein's activity. Thus, “conservatively modified variations” of a particular amino acid sequence refers to amino acid substitutions of those amino acids that are not critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing functionally similar amino acids are well-known in the art. See, e.g., Creighton (1984) Proteins, W. H. Freeman and Company. In addition, individual substitutions, deletions or additions which alter, add or delete a single amino acid or a small percentage of amino acids in an encoded sequence are also “conservatively modified variations.”

The term “stringent conditions” refers to conditions under which a probe or primer will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. In other instances, stringent conditions are chosen to be about 20° C. or 25° C. below the melting temperature of the sequence and a probe with exact or nearly exact complementarity to the target. As used herein, the melting temperature is the temperature at which a population of double-stranded nucleic acid molecules becomes half-dissociated into single strands. Methods for calculating the Tₘ of nucleic acids are well known in the art (see, e.g., Berger and Kimmel (1987) Methods in Enzymology, vol. 152: Guide to Molecular Cloning Techniques, San Diego: Academic Press, Inc. and Sambrook et al. (1989) Molecular Cloning: A Laboratory Manual, 2nd ed., vols. 1-3, Cold Spring Harbor Laboratory), both incorporated herein by reference. As indicated by standard references, a simple estimate of the Tₘ value can be calculated by the equation: Tₘ=81.5+0.41(% G+C), when a nucleic acid is in aqueous solution at 1 M NaCl (see e.g., Anderson and Young, “Quantitative Filter Hybridization,” in Nucleic Acid Hybridization (1985)). Other references include more sophisticated computations which take structural as well as sequence characteristics into account for the calculation of Tₘ. The melting temperature of a hybrid (and thus the conditions for stringent hybridization) is affected by various factors such as the length and nature (DNA, RNA, base composition) of the probe and nature of the target (DNA, RNA, base composition, present in solution or immobilized, and the like), and the concentration of salts and other components (e.g., the presence or absence of formamide, dextran sulfate, polyethylene glycol). The effects of these factors are well known and are discussed in standard references in the art, see e.g., Sambrook et al., Molecular Cloning: A Laboratory Manual, 3rd edition, Cold Spring Harbor Press, N.Y., (2001); Current Protocols in Molecular Biology, (Ausubel, F. M. et al., eds.), John Wiley & Sons, Inc., New York (1987-1993). Typically, stringent conditions will be those in which the salt concentration is less than about 1.0 M Na ion, typically about 0.01 to 1.0 M Na ion concentration (or other salts) at pH 7.0 to 8.3 and the temperature is at least about 30° C. for short probes or primers (e.g., 10 to 50 nucleotides) and at least about 60° C. for long probes or primers (e.g., greater than 50 nucleotides). Stringent conditions can also be achieved with the addition of destabilizing agents such as formamide.

The term “isolated,” “purified” or “substantially pure” means an object species (e.g., an Arp2/3 complex) is the predominant macromolecular species present (i.e., on a molar basis it is more abundant than any other individual species in the composition), and preferably the object species comprises at least about 50 percent (on a molar basis) of all macromolecular species present. Generally, an isolated, purified or substantially pure Arp2/3 complex or nucleic acid will comprise more than 80 to 90 percent of all macromolecular species present in a composition. Most preferably, the object species is purified to essential homogeneity (i.e., contaminant species cannot be detected in the composition by conventional detection methods) wherein the composition consists essentially of a single macromolecular species.

The term “naturally-occurring” as applied to an object refers to the fact that an object can be found in nature. For example, a polypeptide or polynucleotide sequence that is present in an organism that can be isolated from a source in nature and which has not been intentionally modified by humans in the laboratory is naturally-occurring.

“Modulate” can mean either an increase or decrease in the level or magnitude of an activity or process. The increase or decrease can be determined by comparing an activity (e.g., actin polymerization) under a set of test conditions as compared to the activity in a control.

The term “Arp2/3 complex” (or simply Arp2/3) includes its general meaning in the art and includes Arp2/3 from essentially any source (e.g., human, amoeba and budding yeast) that has actin nucleating activity. The term thus refers, for example, to the complex of six subunits in Saccharomyces cerevisiae and seven subunits in Acanthaemoeba castellanii and humans that can nucleate new actin filaments and cross-link newly formed filaments into Y-branched actin filament arrays. Additional details regarding the nomenclature and composition of Arp2/3 complexes from non-human sources are provided in Higgs and Pollard (Ann. Rev. Biochem. 70:649-76 (2001)) and in Welch and Mullins (Annu. Rev. Cell Dev. Biol. 18:247-88, 2002). The term Arp2/3 as used herein encompasses complexes in which one, some or all of the subunits is/are fragments that retain activity, or a variant with substantial sequence identity to a full length sequence or fragment that also has nucleation activity.

An “upstream regulator” as used herein refers includes its general meaning in the art and refers generally to an agent (protein or non-protein) that can activate the activity of a NPF such as WASP and N-WASP so it in turn can activate an actin nucleator such as Arp2/3. Examples of upstream regulators include, but are not limited to (GenBank accession numbers in parentheses): Cdc42 (P21181), TCL and TC10 (Q9H4E5 and P17081), Rac1 (P15154), RhoA (P06749), RhoC (P08134), IRS53 (BAC57946), PAK (Q13153), phosphitydlinositol-1,4-diphosphate (PIP₂), Nck1 (P16333), Nck2 (O43639), Grb2 (P29354), Btk/Itk, WIP (O43516), WICH (JC7807), IcsA (CAC05837), Src kinases (P12931), Hck (P0863 1), Fyn (P06241), CARMIL/Acan 125 (AAK72255), Myosin I (Q9UBC5), PIR121, Nap125, HSPC3000 (AAF28978), EPLIN-inhibitor, IRS53 and Intersectin (Q15811). The upstream regulator, if a protein, can be a full-length naturally occurring protein, a fragment thereof that retains its ability to activate a NPF (e.g., WASP and N-WASP), or a variant that has substantial sequence similarity to a full length protein or fragment and that can activate a NPF.

II. Overview

A variety of WASP and N-WASP proteins are provided that can be expressed in a variety of expression systems and that are soluble in aqueous solution. Some of the WASP and N-WASP proteins that are provided are variants/analogues that have some of the activities associated with full length WASP or N-WASP. Other WASP and N-WASP analogues that are provided can fully recapitulate the activity of full-length naturally occurring forms of WASP and N-WASP. The ability to express full-length WASP and N-WASP in soluble and active form differs from some previous attempts to express full-length WASP, which yielded protein that was insoluble, that could not be regulated by upstream regulators such as Cdc42 and/or was not autoinhibited (see, e.g., Yarar, D., et al. (1999) Curr. Biol. 9:555-558; and Higgs and Pollard (2000) J. Cell. Biol. 150:1311-20). Nucleic acids that encode the WASP and N-WASP proteins are also disclosed, as are cells that contain the nucleic acids.

Methods for expressing the proteins to obtain active and soluble WASP and N-WASP proteins are also disclosed, including methods to express full-length WASP and N-WASP. Purification methods to obtain pure WASP and N-WASP proteins from the expression systems are also provided.

Some of these WASP and N-WASP proteins can be utilized in a variety of applications. For example, the proteins can be used as components in actin polymerization assays to screen libraries of compounds to identify those that modulate the activity of components involved in the actin polymerization pathway. Active compounds so identified can be utilized as candidates in the treatment of various diseases associated with actin polymerization and cell motility (e.g., autoimmune diseases, inflammatory diseases and metastatic cancers). Some of the proteins can also be utilized as inhibitors of the actin polymerization. Certain proteins can also be utilized as the affinity ligand of an affinity chromatography matrix.

III. WASP and N-WASP Proteins

A. General

The term “WASP protein” as used herein refers generally to a protein having an amino acid sequence of a naturally occurring WASP, as well as variants and modified forms regardless of origin or mode of preparation. Similarly, the term “N-WASP protein” encompasses proteins that have an amino acid sequence of a naturally occurring N-WASP and variants regardless of origin or mode of preparation. The WASP or N-WASP protein can be from various sources, including for example, various mammalian and non-mammalian sources.

A naturally occurring or native WASP or N-WASP protein is a protein having the same amino acid sequence as a WASP or N-WASP protein as obtained from nature, respectively. Native sequence WASP and N-WASP proteins specifically encompass naturally occurring truncated or soluble forms, naturally occurring variant forms (e.g., alternatively spliced forms), naturally occurring allelic variants, and forms including posttranslational modifications of WASP and N-WASP, respectively. One specific example of a native sequence of WASP is the full-length native sequence of WASP comprising the amino acid residues as set forth in SEQ ID NO:2. This protein is encoded by the exemplary nucleic acid having the sequence set forth in SEQ ID NO:1. An exemplary native sequence of N-WASP is shown in SEQ ID NO:4, which is encoded by a sequence such as SEQ ID NO:3.

The term “variant” or “analogue” generally refers to proteins that are functional equivalents to a native sequence that have similar amino acid sequences and retain, to some extent, one of the activities of the corresponding native protein. Variants/analogues include fragments that retain one or more activities of the corresponding native protein. Examples of WASP and N-WASP activity include, but are not limited to, capacity to: 1) bind Arp2/3 and actin, 2) activate the actin nucleation activity of Arp2/3 (descriptions of assays to detect nucleation activity are provided below), 3) bind an upstream regulator, 4) be regulated by one or more upstream regulators, thereby rendering the WASP or N-WASP protein able to activate the nucleation activity of Arp2/3, and 5) bind downstream regulators initiating signal transduction cascades. Some of the WASP and N-WASP proteins that are provided are able to recapitulate the full activity of WASP or N-WASP, which means that these proteins have all five of the activities just listed.

Variants and analogues also include proteins that have substantial sequence identity to a corresponding native sequence. Such variants include proteins having amino acid alterations such as deletions, insertions and/or substitutions. A “deletion” refers to the absence of one or more amino acid residues in the related protein. The term “insertion” refers to the addition of one or more amino acids in the related protein. A “substitution” refers to the replacement of one or more amino acid residues by another amino acid residue in the polypeptide. Typically, such alterations are conservative in nature such that the activity of the variant protein is substantially similar to a native sequence WASP or N-WASP (see, e.g., Creighton (1984) Proteins, W. H. Freeman and Company). In the case of substitutions, the amino acid replacing another amino acid usually has similar structural and/or chemical properties. The variations can be made using methods known in the art such as site-directed mutagenesis (Carter, et al. (1986) Nucl. Acids Res. 13:4331; Zoller et al. (I 987) Nucl. Acids Res. 10:6487), cassette mutagenesis (Wells et al. (1985) Gene 34:315), restriction selection mutagenesis (Wells, et al. (1986) Philos. Trans. R. Soc. London SerA 317:415), and PCR mutagenesis (Sambrook, et al. (1989) Molecular Cloning, Cold Spring Harbor Laboratory Press).

Variants of WASP or N-WASP also include modified proteins in which one or more amino acids of a native sequence WASP or N-WASP, respectively, have been altered to a non-naturally occurring amino acid residue. Such modifications can occur during or after translation and include, but are not limited to, phosphorylation, glycosylation, cross-linking, acylation and proteolytic cleavage. Variants also include modified forms in which the protein includes modified protein backbones (e.g., glycosylation, carboxylations, acetylations, ubiquitinization and phosphorylation).

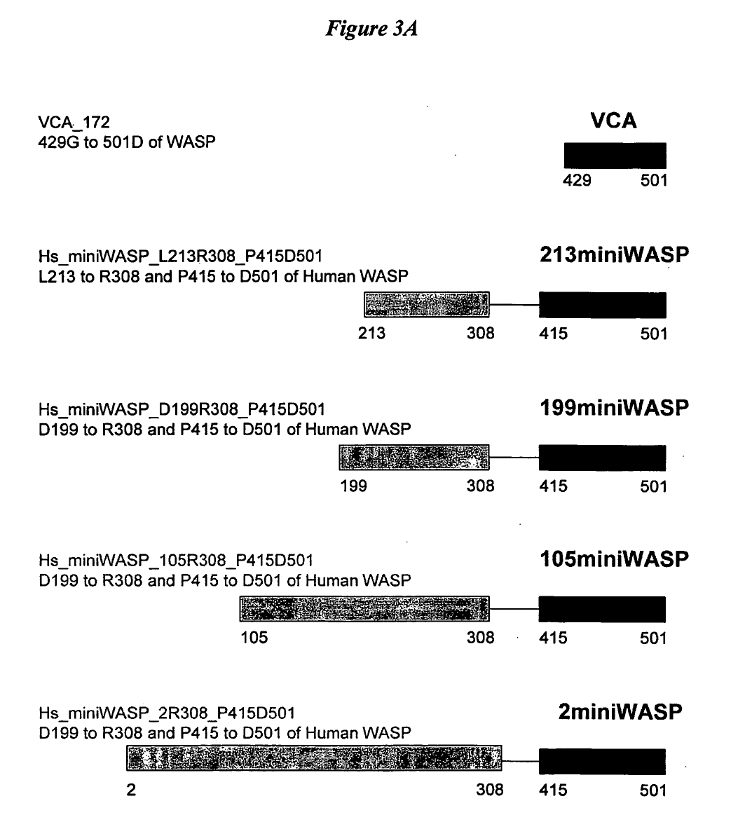

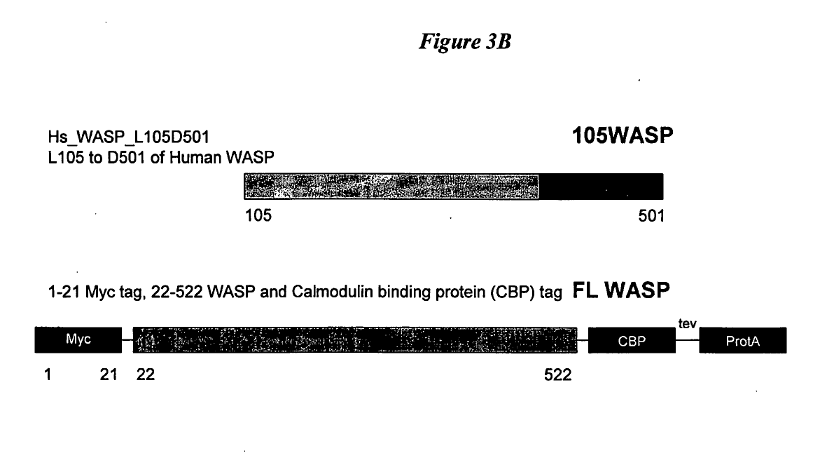

The WASP and N-WASP proteins that are provided generally include both deletion mutants of WASP and N-WASP in which one or more domains have been at least partially deleted (see, e.g., FIGS. 3A and 3B) and fusion proteins that can include: 1) a full length WASP or N-WASP domain or a domain corresponding to the deletion mutant, and 2) one or more tags. The deletion mutants can vary in size, but in some instances are less than 450, 400, 350, 300, 250, 200, 150 or 100 amino acids in length. Typically, the deletion mutants are at least 50, 60, 70 or 80 amino acids in length.

B. Deletion Mutants

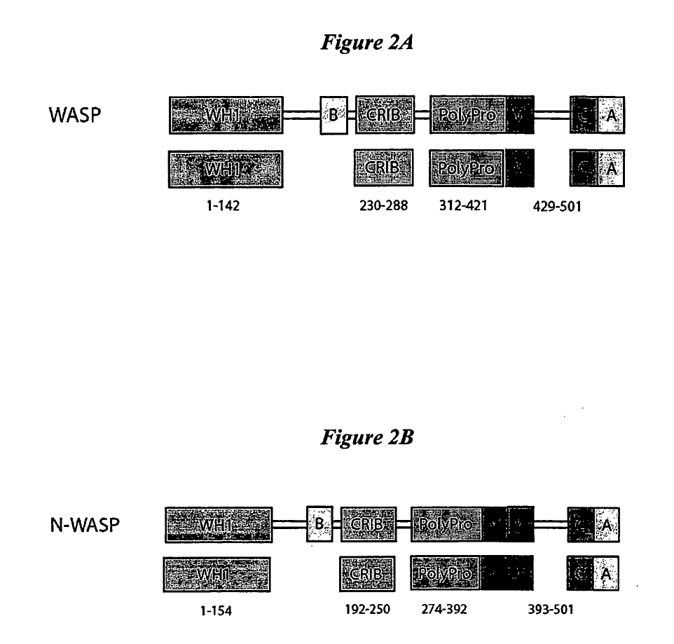

FIGS. 2A and 2B indicate the general organization of the major WASP and N-WASP domains with respect to one another and provides an indication of the approximate boundaries of each of the domains with respect to a full-length WASP sequence (SEQ ID NO:2) and with respect to a full-length N-WASP sequence (SEQ ID NO:4). These regions are also summarized below in Table 1. See also Yarar, D. (2002) Molecular Biology of the Cell 13:4045-59, and Hufner, K. et al. (2001) J. Biol. Chem. 276:35761-7.

It should be recognized, however, that the regions as defined in FIGS. 2A and 2B and Table 1 are approximate and that the regions can extend or omit a limited number of amino acids from the amino or carboxyl end of each domain. For the smaller domains such as the B, CRIB and VCA domains, for instance, the regions can extend or omit about 1, 2, 3, or 4 amino acids from one or both of the amino and carboxyl ends. For the larger domains such as the WH1 domain and the PolyPro domain, the regions can extend or omit about 1-10 amino acids (e.g., 1, 2, 4, 6, 8 or 10) from the amino or carboxyl ends.

One class of deletion mutants/analogues that are provided are WASP and N-WASP proteins in which the WH-1 domain, B domain, CRIB domain (also known as the GTPase Binding Domain (GBD)), and PolyPro domain are disabled. The term “disabled” as used herein with respect to a domain means that the a sufficient part of the domain has been deleted or otherwise affected such that the domain no longer maintains one, some, or all of its activities. In some instances, the entire region encoding the domain is deleted.

A group of proteins in this particular group of deletion mutants are those that include primarily the VCA region. Specific examples of such deletion mutants are proteins that include just the VCA region of WASP (amino acids 429-501 of SEQ ID NO:2; also represented as SEQ ID NO:6) or N-WASP (amino acids 393-501 of SEQ ID NO:4; also represented as SEQ ID NO:8).

A second class of WASP and N-WASP proteins are those in which the WH-1 and PolyPro region have been at least partially removed. Specific examples of WASP and N-WASP proteins lacking at least a part or all of the WH1 and PolyPro regions include, but are not limited to, 213 miniWASP, 199 miniWASP and 105 miniWASP (see, FIGS. 3A and 3B). These three proteins each lack some or all of the WH-1 region, and the entire PolyPro region (approximately residues 309-414 of SEQ ID NO:2). The 213 miniWASP protein thus includes, for example, amino acid residues 213-308 and 415-501 from the full length WASP sequence (SEQ ID NO:2). 199 miniWASP includes residues 199-308 and 415-501 of SEQ ID NO:2). 105 miniWASP includes residues 105-308 and 415-501 of SEQ ID NO:2). 213 miniWASP and 199 miniWASP are regulated by Cdc42 (i.e., Cdc42 can bind and activate the construct so the activated construct can in turn activate the nucleation activity of Arp2/3).

A specific example that lacks only a portion of WH-1 but still lacks the PolyPro region is 2 miniWASP. This particular protein includes residues 2-308 and 415-501 of SEQ ID NO:2.

A third class of WASP and N-WASP proteins that are provided are those which include the WH-1 domain but in which the PolyPro region is disabled (e.g., deleted).

A fourth class of WASP and N-WASP proteins that are provided are deletion mutants in which the PolyPro region is maintained but an N-terminal region (e.g., WH1 domain) is at least partially removed. Some proteins in this class are ones that include the B, CRIB/GBD, PolyPro and VCA domains but in which some or all of the WH1 has been removed. One specific example is 98N-WASP, which includes amino acids 98-501 of SEQ ID NO:4 (also assigned SEQ ID NO:12). Another specific example is the 105 WASP protein, which includes amino acids 105-501 of SEQ ID NO:10 (see FIG. 3A and 3B). Both 98N-WASP and 105 WASP protein are of interest because they fully recapitulate the activity of full-length WASP in that they are regulated by Cdc42, PIP₂, Nck1, and Rc1. They can also activate actin nucleation by Arp2/3.

Table 2 summarizes the sequences of these specific constructs and indicates whether the protein can activate the nucleation activity of Arp2/3 and whether the protein can be regulated by the upstream regulators Cdc42, PIP₂, Nck and Rac1.

The proteins that are provided also include variants of the foregoing four classes of proteins that have substantial sequence identity to the proteins in these classes and that retain some or all of the same activities. The WASP and N-WASP proteins that are provided thus include, for example, proteins that have substantial sequence identity with SEQ ID NOs:2, 4, 6, 8, 10 and 12 and that retain the activity of the corresponding protein as listed in Table 2.

C. WASP and N-WASP Fusion Proteins

The WASP and N-WASP proteins that are provided can also be fusion proteins. Such fusion proteins in general include: 1) a WASP or N-WASP domain, which can be a full length WASP or N-WASP sequence (e.g., SEQ ID NOs:2 and 4, respectively) or an analogue/deletion mutant such as described above (e.g., SEQ ID NOs:6, 8, 10, 12), and 2) one or more tag domains linked or fused to the amino and/or carboxyl terminal ends of the WASP or N-WASP protein domain. The fusion proteins thus also include fusion proteins that result from the removal of a tag from either the amino or carboxy terminus of a fusion protein that initially included a tag at each end. Some of the fusion proteins are of interest because they have the same activities of naturally occurring WASP and N-WASP.

The tags that are incorporated into the fusion can be utilized to improve expression, to improve solubility and/or to aid in purification. The WASP or N-WASP domain can also be a protein that has substantial sequence identity to full-length WASP or N-WASP or the various deletion mutants listed above. Thus, for example, the WASP or N-WASP domain can have substantial sequence identity to SEQ ID NO:s 2, 4, 6, 8, 10 and 12.

A variety of tags can be utilized, including but are not limited to: 1) a glutathione S-transferase (GST) tag, which can be used to bind to glutathione-agarose; 2) a His6 tag (or simply a HIS tag), which can be used to bind to immobilized metal-ion columns (e.g., nickel); 3) a calmodulin-binding peptide (CBP) tag that binds calmodulin-agarose columns; 4) an epitope tag (e.g., a haemagglutinin tag, a myc tag, or a FLAG tag), which can be used to bind an antibody with specific binding affinity for the epitope tag; 5) a maltose-binding protein (MBP) tag, which increases the solubility of fused proteins; and 5) a TAP tag, which the current inventors have determined can be utilized to facilitate expression of WASP and N-WASP proteins and to improve their solubility. These tags can also be used in combination, with one or more tags fused to the amino terminus and one or more additional tags fused to the carboxyl terminus.

Many of these tags are commercially available. For example, vectors useful for incorporating HIS tags in mammalian cells include vectors pcDNA3.1/Myc-His and pcDNA3.1/V5-His, which are available from Invitrogen (Carlsbad, Calif.). Vectors pBlueBacHis and Gibco (Gaithersburg, Md.) vectors pFastBacHT are suitable for expression in insect cells. HIS tags and their use with metal chelate affinity ligands such as nitrilo-tri-acetic acid (NTA) that can bind the poly histidine tag are discussed, for example, by Hochuli (“Purification of recombinant proteins with metal chelating adsorbents” In Genetic Engineering: Principles and Methods, J. K. Setlow, Ed., Plenum Press, NY, 1990). Systems for incorporating His tags are available from Qiagen. FLAG tags are discussed by, for example, Chubet and Brizzard (Biotechniques 20:136-141, 1996), and Knappik and Pluckthun (Biotechniques 17:754-761, 1994). Systems for fusing a GST tags are available, for example, from Promega. New England Biolabs provides systems for incorporating MBP tags. CBP systems can be obtained from Strategene. FLAG tags to a protein are available from various sources, including Kodak, Rochester N.Y.

Tags such as these can optionally be linked to segments that include protease cleavage sites to facilitate removal of the purification tag and to simultaneously elute the proteins. An example are fusion proteins in which the WASP or N-WASP protein is linked to a tag via a linker that includes a protease cleavage site such as the tobacco etch virus (TEV) protease site. The tag can be used to bind to a column that includes an appropriate ligand to bind the tag. The bound fusion protein can subsequently be released by exposing the column to a highly specific TEV protease. Further details regarding such a strategy are described in the examples. See also, Carrington and Dougherty (1988) Proc. Natl. Acad. Sci. USA 85: 3391-3395; Dougherty, et al. (1989) Virology 171: 356-364; Dougherty and Semler (1993) Microbiol. Rev. 57: 781-822; Herskovits, et al. (2001) EMBO Reports 2:1040-1046; Ehrmann, et al. Proc. Natl. Acad. Sci. USA 94:13111-13115; Faber et al. (2001) J. Biol. Chem. 276: 36501-36507; Smith and Kohorn (1991) Proc. Natl. Acad. Sci. USA. 88: 5159-5162; Kapust et al. (2001) Protein Eng. 14:993-1000; and Melcher (2000) Anal Biochem 277:109-120.

One specific example are TAP tags that include a TEV cleavable site. TAP tags generally include an IgG-binding unit from Protein A of Staphyloccoccus (ProtA) and a binding unit from Calmodulin Binding Peptide (CBP). Certain TAP tags that are useful for fusing to the C-terminus of a protein are part of a construct that includes CBP, a TEV cleavage site and ProtA (SEQ ID NO:36, encoded for example by SEQ ID NO:35). Strategies for using TAP tags in certain applications are discussed, for instance, by Rigaut, et al. (Nature Biotechnology 17:1030-1032, 1999) and Puig, et al. (Yeast 14:1139-1146), both of which are incorporated herein by reference in its entirety for all purposes.

Specific examples of fusion proteins that are provided include those that include a segment that encode full-length WASP or N-WASP, including: Myc-WASP-TAP (SEQ ID NO:14), Myc-N-WASP-TAP (SEQ ID NO:16). Examples of fusion proteins that include a WASP or N-WASP deletion mutant domain include: GST-105WASP (SEQ ID NO:18), Myc-105 WASP-TAP (SEQ ID NO:20), GST-tev-98N-WASP (SEQ ID NO:22), and Myc-98N-WASP-TAP (SEQ ID NO:24). The TAP tag in these particular fusion proteins has the general structure CBP-tev-ProtA (SEQ ID NO:36). Fusion proteins such as those listed in Table 2 can in some instances be utilized directly, or after one or both of the C- and N-terminal tags are removed. For example, the fusion proteins listed in Table 2 as having a TAP tag can be used once the TAP tag has been cleaved off and/or after the C-terminal tag has been removed.

Details regarding the preparation of some of the full length NPF proteins, miniWASPs and other WASP fragments that are fused to tags are provided in the examples below. Other fusion proteins containing one or more tags can be prepared using conventional molecular biological techniques such as described in Sambrook et al. Molecular Cloning: A Laboratory Manual, 3rd ed., Cold Spring Harbor Press, N.Y. (2001); and Current Protocols in Molecular Biology (Ausubel, F. M., et al. eds.), John Wiley & Sons, Inc., New York (1987-1993), which are incorporated herein by reference in their entirety for all purposes.

IV. Nucleic Acids

A. Exemplary Sequences

Nucleic acids that encode the WASP and N-WASP proteins described above are also provided. Nucleic acids encoding fusion proteins that include a WASP or N-WASP domain corresponding to full-length WASP or N-WASP or a deletion mutant such as described herein are also provided.

Thus, one set of nucleic acids include those that encode the deletion mutants or analogues in which the WH-1, B, CRIB/GBD and PolyPro domains have been disabled. Exemplary nucleic acids thus include those that encode for the VCA domains corresponding to amino acids 429-501 of SEQ ID NO:2 and those that encode for VCA domains corresponding to amino acids 393-501 of SEQ ID NO:4. Exemplary nucleic acid sequences encoding such proteins include SEQ ID NOs:1 and 3, respectively.

The nucleic acids that are provided also include those that encode for WASP and N-WASP proteins in which the WH-1 and PolyPro region have been disabled. The nucleic acids in this group thus include those that encode for 213 miniWASP, 199 miniWASP and 105 miniWASP.

Other nucleic acids encode for WASP and N-WASP proteins in which the WH-1 domain is included but in which the PolyPro region is disabled (e.g., deleted).

Still other nucleic acids that are provided are those that encode for WASP and N-WASP proteins that are deletion mutants in which the polypro region is maintained but an N-terminal region (e.g., WH1 domain) is disabled or deleted. Such nucleic acids thus encode, for example, 105 WASP protein (SEQ ID NO:10) and 98N-WASP (SEQ ID NO:12). Specific examples of such nucleic acids include SEQ ID NOs:9 and 11, respectively.

Nucleic acids that encode for the various fusion proteins that are described herein are also provided. The provided nucleic acids thus include, for example, those that encode the full-length sequence of WASP or N-WASP and one or more tags from those listed above that are linked to the carboxyl and/or amino terminal end of the WASP or N-WASP sequence. Examples of such nucleic acids include those that encode the Myc-WASP-TAP fusion protein (SEQ ID NO:14) and the nucleic acids that encode the Myc-N-WASP-TAP fusion (SEQ ID NO:16). Exemplary nucleic acids encoding the Myc-WASP-TAP fusion include SEQ ID NO:13; exemplary nucleic acids encoding the Myc-N-WASP-TAP fusion protein include SEQ ID NO:15.

Additional specific examples of nucleic acids that are provided include those that encode the GST-105 WASP fusion (SEQ ID NO:18), the Myc-105WASP-TAP fusion (SEQ ID NO:20), the GST-tev-98N-WASP fusion (SEQ ID NO:22); and the Myc-98N-WASP-TAP fusion protein (SEQ ID NO:24). Specific examples of the nucleic acids that encode these fusions are listed in Table 2.

The nucleic acids that are provided include not just the exemplary nucleic acids listed herein as encoding the various disclosed WASP and N-WASP proteins (e.g., the deletion mutants and fusion proteins), but all other nucleic acids that encode these proteins but differ from the listed sequences due to the degeneracy of the genetic code. The nucleic acids that are provided also include nucleic acids that are complementary to the listed sequences. Additionally, the nucleic acids include those that have substantial sequence identity to the nucleic acids that are described herein, provided the nucleic acids encode a protein that has an activity associated with WASP (e.g., ability to bind Arp2/3, ability to activate the nucleation activity of Arp2/3, ability to bind an upstream regulator and/or the ability to be activated by an upstream regulator).

B. Obtaining Nucleic Acids

A number of different approaches can be utilized to obtain the nucleic acids that are provided, including, for example: 1) hybridization of genomic or cDNA libraries with probes to detect homologous nucleotide sequences; 2) various amplification procedures such as polymerase chain reaction (PCR) using primers capable of annealing to the nucleic acid of interest; and 3) direct chemical synthesis.

Full-length WASP and N-WASP, for example, can be isolated using probes that specifically hybridize to a WASP or N-WASP sequence in a cDNA library, a WASP or N-WASP gene in a genomic DNA sample, or to a WASP or N-WASP mRNA in a total RNA sample (e.g., in a Southern or Northern blot). Once the target nucleic acid is identified, it can be isolated according to standard methods known to those of skill in the art.

The desired nucleic acids can also be cloned using well-known amplification techniques. Examples of protocols sufficient to direct persons of skill through in vitro amplification methods, including the polymerase chain reaction (PCR), the ligase chain reaction (LCR), Qβ-replicase amplification and other RNA polymerase mediated techniques, are found in Berger, Sambrook, and Ausubel, as well as Mullis et al. (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87: 1874; Lomell et al. (1989) J. Clin. Chem. 35: 1826; Landegren et al. (1988) Science 241: 1077-1080; Van Brunt (1990) Biotechnology 8: 291-294; Wu and Wallace (1989) Gene 4: 560; and Barringer et al. (1990) Gene 89: 117. Improved methods of cloning in vitro amplified nucleic acids are described in Wallace et al., U.S. Pat. No. 5,426,039. Suitable primers for use in the amplification of some of the nucleic acids of the invention are provided in Examples 1 and 2.

Nucleic acids encoded the desired WASP or N-WASP proteins can also be chemically synthesized. Direct chemical synthesis methods include, for example, the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. One of skill would recognize that while chemical synthesis of DNA is often limited to sequences of about 100 bases, longer sequences may be obtained by the ligation of shorter sequences. Alternatively, subsequences may be cloned and the appropriate subsequences cleaved using appropriate restriction enzymes. The fragments can then be ligated to produce the desired DNA sequence.

If it is desired to modify the nucleic acids that are disclosed herein, this can be accomplished using a variety of established techniques. Examples of such methods include, for instance, site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques. See, e.g., Gilman and Smith (1979) Gene 8:81-97, Roberts et al. (1987) Nature 328: 731-734.

V. Methods of Preparing WASP and N-WASP Proteins

A. General

The nucleic acid sequences that are provided can be utilized in the production of the WASP and N-WASP proteins that are provided using various recombinant techniques. For example, the cloned DNA sequences can be expressed in hosts after the sequences have been operably linked to an expression control sequence in an expression vector. Expression vectors are typically replicable in the host organisms either as episomes or as an integral part of the host chromosomal DNA. Commonly, expression vectors contain selection markers, e.g., tetracycline resistance or hygromycin resistance, to permit detection and/or selection of those cells transformed with the desired DNA sequences (see, e.g., U.S. Pat. No. 4,704,362).

B. Expression Cassettes and Host Cells for Expressing Polypeptides

Typically, a nucleic acid that encodes a WASP or N-WASP protein is placed under the control of a promoter that is functional in the desired host cell to produce relatively large quantities of the WASP or N-WASP protein of interest. A wide variety of promoters can be used in the expression vectors, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Accordingly, expression cassettes are provided into which the nucleic acids that encode the WASP and N-WASP proteins are incorporated for high level expression in a desired host cell.

The WASP and N-WASP proteins that are deletion mutants can be expressed in a variety of systems, including both prokaryotic and eurkaryotic systems such as those described below (see, also Examples 4 and 5). The WASP and N-WASP nucleic acids that encode full-length sequences are typically expressed in eukaryotic cells, including human cells lines such as 293 cells.

Certain expression cassettes are useful for expression of the polypeptides of the invention in prokaryotic host cells. Commonly used prokaryotic control sequences, which are defined herein to include promoters for transcription initiation, optionally with an operator, along with ribosome binding site sequences, include such commonly used promoters as the beta-lactamase (penicillinase) and lactose (lac) promoter systems (Change et al. (1977) Nature 198: 1056), the tryptophan (trp) promoter system (Goeddel et al. (1980) Nucleic Acids Res. 8: 4057), the tac promoter (DeBoer et al. (1983) Proc. Natl. Acad. Sci. U.S.A. 80:21-25); and the lambda-derived P_{L} promoter and N-gene ribosome binding site (Shimatake et al. (1981) Nature 292: 128). The particular promoter system is not critical, any available promoter that functions in prokaryotes can be used.

For expression of proteins in prokaryotic cells other than E. coli, a promoter that functions in the particular prokaryotic species is required. Such promoters can be obtained from genes that have been cloned from the species, or heterologous promoters can be used. For example, the hybrid trp-lac promoter functions in Bacillus in addition to E. coli.

For expression of the polypeptides in yeast, convenient promoters include GAL1-10 (Johnson and Davies (1984) Mol. Cell. Biol. 4:1440-1448), ADH2 (Russell et al. (1983) J. Biol. Chem. 258:2674-2682), PHO5 (EMBO J. (1982) 6:675-680), and MFα (Herskowitz and Oshima (1982) in The Molecular Biology of the Yeast Saccharomyces (eds. Strathern, Jones, and Broach) Cold Spring Harbor Lab., Cold Spring Harbor, N.Y., pp. 181-209). Another suitable promoter for use in yeast is the ADH2/GAPDH hybrid promoter as described in Cousens et al., Gene 61:265-275 (1987). Other promoters suitable for use in eukaryotic host cells are well-known to those of skill in the art.

For expression of the polypeptides in mammalian cells, convenient promoters include CMV promoter (Miller, et al., BioTechniques 7:980), SV40 promoter (de la Luma, et al., (1998) Gene 62:121), RSV promoter (Yates, et al., (1985) Nature 313:812), and MMTV promoter (Lee, et al., (1981) Nature 294:228).

For expression of the polypeptides in insect cells, the convenient promoter is from the baculovirus Autographa Californica nuclear polyhedrosis virus (NcMNPV) (Kitts, et al., (1993) Nucleic Acids Research 18:5667).

Either constitutive or regulated promoters can be used. Regulated promoters can be advantageous because the host cells can be grown to high densities before expression of the polypeptides is induced. High level expression of heterologous proteins slows cell growth in some situations. An inducible promoter is a promoter that directs expression of a gene where the level of expression is alterable by environmental or developmental factors such as, for example, temperature, pH, anaerobic or aerobic conditions, light, transcription factors and chemicals. Such promoters are referred to herein as “inducible” promoters, and allow one to control the timing of expression of the polypeptide. For E. coli and other bacterial host cells, inducible promoters are known to those of skill in the art. These include, for example, the lac promoter, the bacteriophage lambda P_{L} promoter, the hybrid trp-lac promoter (Amann et al. (1983) Gene 25: 167; de Boer et al. (1983) Proc. Natl. Acad. Sci. USA 80: 21), and the bacteriophage T7 promoter (Studier et al. (1986) J. Mol. Biol.; Tabor et al. (1985) Proc. Natl. Acad. Sci. USA 82: 1074-8). These promoters and their use are discussed in Sambrook et al., supra. A particularly preferred inducible promoter for expression in prokaryotes is a dual promoter that includes a tac promoter component linked to a promoter component obtained from a gene or genes that encode enzymes involved in galactose metabolism (e.g., a promoter from a UDP galactose 4-epimerase gene (galE)). The dual tac-gal promoter, which is described in PCT Patent Application Publ. No. WO98/20111, provides a level of expression that is greater than that provided by either promoter alone.

Inducible promoters for other organisms are also well-known to those of skill in the art. These include, for example, the arabinose promoter, the lacZ promoter, the metallothionein promoter, and the heat shock promoter, as well as many others.

A ribosome binding site (RBS) is conveniently included in the expression cassettes that are intended for use in prokaryotic host cells. An RBS in E. coli, for example, consists of a nucleotide sequence 3-9 nucleotides in length located 3-11 nucleotides upstream of the initiation codon (Shine and Dalgarno (1975) Nature 254: 34; Steitz, In Biological regulation and development: Gene expression (ed. R. F. Goldberger), vol. 1, p. 349, 1979, Plenum Publishing, NY).

Selectable markers are often incorporated into the expression vectors used to express the polynucleotides of the invention. These genes can encode a gene product, such as a protein, necessary for the survival or growth of transformed host cells grown in a selective culture medium. Host cells not transformed with the vector containing the selection gene will not survive in the culture medium. Typical selection genes encode proteins that confer resistance to antibiotics or other toxins, such as ampicillin, neomycin, kanamycin, chloramphenicol, or tetracycline. Alternatively, selectable markers can encode proteins that complement auxotrophic deficiencies or supply critical nutrients not available from complex media, e.g., the gene encoding D-alanine racemase for Bacilli. Often, the vector will have one selectable marker that is functional in, e.g., E. coli, or other cells in which the vector is replicated prior to being introduced into the host cell. A number of selectable markers are known to those of skill in the art and are described for instance in Sambrook et al., supra. A preferred selectable marker for use in bacterial cells is a kanamycin resistance marker (Vieira and Messing, Gene 19: 259 (1982)). Use of kanamycin selection is advantageous over, for example, ampicillin selection because ampicillin is quickly degraded by β-lactamase in culture medium, thus removing selective pressure and allowing the culture to become overgrown with cells that do not contain the vector.

Construction of suitable vectors containing one or more of the above listed components employs standard ligation techniques as described in the references cited above. Isolated plasmids or DNA fragments are cleaved, tailored, and re-ligated in the form desired to generate the plasmids required. To confirm correct sequences in plasmids constructed, the plasmids can be analyzed by standard techniques such as by restriction endonuclease digestion, and/or sequencing according to known methods. A wide variety of established cloning and in vitro amplification methods suitable for the construction of recombinant nucleic acids can be utilized. Examples of these techniques and instructions sufficient to direct persons of skill through many cloning exercises are found in Berger and Kimmel, Guide to Molecular Cloning Techniques, Methods in Enzymology, Volume 152, Academic Press, Inc., San Diego, Calif. (Berger); and Current Protocols in Molecular Biology, F. M. Ausubel et al., eds., Current Protocols, a joint venture between Greene Publishing Associates, Inc. and John Wiley & Sons, Inc. (1998 Supplement) (Ausubel).

A variety of common vectors suitable for use as starting materials for constructing the expression vectors of the invention are well-known in the art. For cloning in bacteria, common vectors include pBR322 derived vectors such as pBLUESCRIPT™, pUC18/19, and λ-phage derived vectors. In yeast, vectors which can be used include Yeast Integrating plasmids (e.g., YIp5) and Yeast Replicating plasmids (the YRp series plasmids) pYES series and pGPD-2, for example. Expression in mammalian cells can be achieved, for example, using a variety of commonly available plasmids, including pSV2, pBC12BI, and p91023, pCDNA series, pCMV1, pMAMneo, as well as lytic virus vectors (e.g., vaccinia virus, adeno virus), episomal virus vectors (e.g., bovine papillomavirus), and retroviral vectors (e.g., murine retroviruses). Expression in insect cells can be achieved using a variety of baculovirus vectors, including pFastBac1, pFastBacHT series, pBluesBac4.5, pBluesBacHis series, pMelBac series, and pVL1392/1393, for example.

Translational coupling can be used to enhance expression. The strategy uses a short upstream open reading frame derived from a highly expressed gene native to the translational system, which is placed downstream of the promoter, and a ribosome binding site followed after a few amino acid codons by a termination codon. Just prior to the termination codon is a second ribosome binding site, and following the termination codon is a start codon for the initiation of translation. The system dissolves secondary structure in the RNA, allowing for the efficient initiation of translation. See, Squires et. al. (1988) J. Biol. Chem. 263: 16297-16302.

The WASP and N-WASP proteins that are provided can be expressed in a variety of host cells, including E. coli, other bacterial hosts, yeast, and various higher eukaryotic cells such as the COS, 293, CHO and HeLa cells lines and myeloma cell lines. The host cells can be mammalian cells, plant cells, insect cells or microorganisms, such as, for example, yeast cells, bacterial cells, or fungal cells. Examples of suitable host cells include Azotobacter sp. (e.g., A. vinelandii), Pseudomonas sp., Rhizobium sp., Erwinia sp., Escherichia sp. (e.g., E. coli), Bacillus, Pseudomonas, Proteus, Salmonella, Serratia, Shigella, Rhizobia, Vitreoscilla, Paracoccus and Klebsiella sp., among many others. The cells can be of any of several genera, including Saccharomyces (e.g., S. cerevisiae), Candida (e.g., C. utilis, C. parapsilosis, C. krusei, C. versatilis, C. lipolytica, C. zeylanoides, C. guilliermondii, C. albicans, and C. humicola), Pichia (e.g., P. farinosa and P. ohmeri), Torulopsis (e.g., T. candida, T. sphaerica, T. xylinus, T. famata, and T. versatilis), Debaryomyces (e.g., D. subglobosus, D. cantarellii, D. globosus, D. hansenii, and D. japonicus), Zygosaccharomyces (e.g., Z. rouxii and Z. bailii), Kluyveromyces (e.g., K. marxianus), Hansenula (e.g., H. anomala and H. jadinii), and Brettanomyces (e.g., B. lambicus and B. anomalus). Examples of useful bacteria include, but are not limited to, Escherichia, Enterobacter, Azotobacter, Erwinia, Klebsielia. The commonly used insect cells to produce recombinant proteins are Sf9 cells (derived from Spodoptera frugiperda ovarian cells) and High Five cells (derived from Trichoplusia ni egg cell homogenates; commercially available from Invitrogen). Thus, cells containing the nucleic acids that are provided are also included.

The expression vectors of the invention can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

C. Exemplary Expression Systems

As described in greater detail in Examples 5 and 10, WASP and N-WASP proteins that fully recapitulate the activity of native WASP and N-WASP and that are soluble in aqueous solution can be obtained by expressing WASP and N-WASP constructs in human cells such as 293 cells. Exemplary constructs that introduced into the cells to encode such proteins are ones that encode proteins having the general structure WASP-TAP (i.e., WASP-CBD-tev-Prot A;), or N-WASP-TAP (i.e., N-WASP-CBD-tev-ProtA). These constructs can also include a variety of N-terminal tags, including those listed above (e.g., myc). Examples of constructs with both N- and C-terminal tags include Myc-WASP-TAP (SEQ ID NO:14, encoded by SEQ ID NO:13) and Myc-N-WASP-TAP (SEQ ID NO:16, encoded by SEQ ID NO:15).

Additional details regarding exemplary methods for expressing some of the other WASP and N-WASP proteins that are provided are provided in Examples 4 and 5.

VI. Purification of WASP and N-WASP Proteins

The recombinant proteins that are provided herein can be purified utilizing a variety of methods. Once expressed, the recombinant WASP and N-WASP proteins can be purified according to standard procedures of the art, including ammonium sulfate precipitation, affinity columns, ion exchange and/or size exclusivity chromatography, gel electrophoresis and the like (see, generally, R. Scopes, Protein Purification, Springer-Verlag, N.Y. (1982), Deutscher, Methods in Enzymology Vol. 182: Guide to Protein Purification., Academic Press, Inc. N.Y. (1990)).

If the WASP or N-WASP protein includes one or more purification tags, these tags can be utilized to purify the protein according to established techniques. As noted above, systems for preparing fusion proteins that include His, GST, MBP, and CBP are available from Qiagen, Promega, New England Biolabs and Strategene, respectively. These suppliers provide information on the use of such tags as part of a purification strategy. Amersham Biosciences also produces a variety of column chromatographic material that includes the appropriate affinity ligands to bind to these various tags.

Fusion proteins including the TAP tag and a TEV cleavage site can be purified by tandem affinity purification methods. As noted above, a typical fusion protein including a TAP tag has the general structure WASP (or N-WASP) protein domain-CBP-TEV-ProtA. Thus, some tandem purification methods generally initially involve recovering the WASP or N-WASP fusion protein by affinity selection on an IgG-matrix, with the ProtA domain of the fusion protein becoming bound to the IgG matrix. After the IgG material has been washed, TEV protease is added to cleave the fusion protein at the TEV cleavage site, thereby releasing the bound fusion protein and leaving a WASP (or N-WASP)-CBP fusion construct. The eluate containing this construct is then typically incubated with calmodulin-coated beads in the presence of calcium. The WASP (or N-WASP)-CBP fusion binds to the beads under these conditions. This allows TEV protease and other contaminants to be washed away. After washing, the fusion bound to the calmodulin beads is released by adding EGTA to bind up the calcium. Additional details are provided in Example 5. See also, Marani, et al. (2002) Mol. Cell Biol. 22:3577-3589.

Using purification schemes such as these, WASP or N-WASP proteins of high purity can be obtained. Some proteins, for instance, are at least 70, 75, 80, 85, 90, 95, 97 or 99% pure.

VII. Exemplary Applications

The WASP and N-WASP proteins that are provided can be utilized in a variety of ways. Some of the proteins, for example, can be utilized as affinity ligands that can be coupled to an affinity matrix material. Because certain of the proteins can bind Arp2/3 and/or upstream regulators, these proteins can be utilized to purify Arp2/3 and/or upstream regulators (e.g., Cdc42, Nck1, Rac2). Methods for coupling affinity ligands to a variety of affinity matrix materials can be utilized (see, e.g., Affinity Chromatography: Principles and Methods, Amersham Pharmacia Biotech AB, 2001). Activated matrix material to which the ligands can be coupled are available from, for example, Amersham Pharmacia Biotech.

Certain of the WASP and N-WASP proteins can also be utilized as inhibitors of naturally occurring WASP and N-WASP proteins. Such proteins can be utilized in various screening methods, for example.

Some of the WASP and N-WASP proteins that are provided can also be used in screening methods to identify agents that modulate the activity of components involved in actin polymerization. The ability to screen for such agents is important because of the important role that actin polymerization plays in many cellular processes, including those that are related to various autoimmune and inflammatory diseases and metastatic cancer. Some screening methods are designed such that the use of the WASP and N-WASP proteins that are provided can be used to identify agents that modulate the activity of actin, WASP or N-WASP, and/or upstream regulators.

Some screening methods in which the WASP and N-WASP proteins that are described herein can be utilized take advantage of the fundamental role that Arp2/3 plays in the formation of branched actin filament networks. These screening methods are also based on the recognition that actin polymerization involves a series of regulated processes in which: 1) an upstream regulator binds WASP or N-WASP to activate it, 2) activated WASP or N-WASP in turn binds Arp2/3 and activates it, 3) Arp2/3 initiates nucleation of actin, and 4) G-actin is further incorporated into the nucleated actin to form F-actin. The formation of F-actin can be detected in various ways but in general involves detecting a characteristic that distinguishes F-actin from G-actin.

The components included in the screening assay typically include G-actin, Arp2/3 or other nucleator protein, a WASP or N-WASP protein that can activate Arp2/3, and/or one or more upstream regulators. Various actin binding proteins can also be included in some assays. Upon addition of suitable polymerization salts, Arp2/3, and NPFs, polymerization occurs following a lag phase related to the spontaneous formation of actin filament seeds. Once sufficient filaments have formed to bind all the available Arp2/3 along their sides, the total rate of G-actin to F-actin conversion is linearly related to the number of filament ends and to the G-actin concentration. Since each activated Arp2/3 molecule generates one filament end, if the Arp2/3 concentration is large enough to render the number of filament ends generated by spontaneous polymerization negligible, the rate of polymerization is linearly related to the concentration of activated Arp2/3.

The screening methods generally involve combining components of an actin polymerization assay together in the presence of a candidate agent under conditions in which, in the absence of the candidate agent, G-actin can become incorporated into F-actin. After the assay components have been combined, polymerization is detected over time to determine a parameter that is a measure of the extent of the polymerization of actin into F-actin. The value for the determined polymerization parameter is then optionally compared with the polymerization parameter determined for a corresponding control assay. A difference between the parameters is an indication that the candidate agent is a modulator of one of the assay components.

In some methods, the polymerization reaction is detected by including pyrene-labeled G-ATP-actin in the assay mixture. The fluorescence spectrum of pyrene-actin changes on polymerization. In F-actin, the pyrene fluorescence is blueshifted and shows an altered lineshape such that the maximum of the F-G difference spectrum occurs at 407 nm but the G-actin fluorescence is more intense at wavelengths above ˜430 nm. Other methods, however, utilize dyes that exhibit considerable fluorescence enhancement in f-actin solutions as compared to G-actin solutions (e.g., the fluorescent dye 4-(dicyano)julolidine (DCVJ)).

Further details regarding the use of certain of the WASP and N-WASP proteins that are disclosed herein are provided in Example 7. Additional details regarding the use of some of the disclosed proteins in screening assays to identify modulators of actin polymerization are provided in U.S. Provisional Application No. 60/578,949, filed Jun. 10, 2004, which is incorporated herein by reference in its entirety for all purposes.

The following examples are offered to illustrate certain aspects of the WASP and N-WASP proteins that are provided and their use in various applications. These examples, however, should not be construed to limit the claimed invention.

### EXAMPLE 1

### Cloning of WASP Proteins

A. Cloning of WASP VCA Region

1. WASP full length cDNA is used as a template to amplify the coding sequence. Oligo (forward): 5′-GGGGACAAGTTTGTACAAAAAAGCAGGCTTCGAAAACCTGTATTTTCAGG GCGGGGGTCGGGGAGCGCTTTTGGATC-3′ (SEQ ID NO:41) and oligo (reverse): 5′-GGGGACCACTTTGTACAAGAAAGCTGGGTCCTAGTCATCCCATTCATCATC TTCATC-3′ (SEQ ID NO:42) are used in the reaction.

2. The pcr fragment is cloned into pDONR201 (Invitrogen Life Technology, Cat# 11798-014) by Gateway BP reaction to generate pDONR_tev_HsWASPVCA.

3. Clone pDONR_tev_HsWASPVCA into pDEST15 (Invitrogen Life Technology, Cat# 11802-014) to generate N-GST_tev_HsWASPVCA by LR Gateway recombination reaction.

B. Cloning of N_GST_{—}105LWASP (Bacterial GST Tagged Protein)

1. WASP full length is used as a template to amplify the coding sequence. Oligo (forward): 5′-CACCGAAAACCTGTATTTTCAGGGCCTTGTCTACTCCACCCCCACCCCC-3′ (SEQ ID NO:43) and oligo (reverse):5′-CTAGTCATCCCATTCATCATCTTC-3′ (SEQ ID NO:44) are used in the reaction.

2. The pcr fragment is cloned into pENTR/SD/TOPO vector (Invitrogen Life Technology, Cat# K2400-20) by directional cloning using Topoisomerase I.

3. The pENTR/SD/TOPO_{—}105LWASP is cloned into pDEST15 (Invitrogen Life Technology, Cat# 11802-014) by Gateway LR reaction to generate N_GST_{—}105LWASP.

C. Cloning of pcDNA3.1Myc_{—}105LWASPTAP (Mammalian TAPTAG Tagged Protein)

1. WASP full length (American Type Culture Collection, Cat# 99534) is used as a template to amplify the coding sequence. Oligo (forward): 5′-GGGGACAAGTTTGTACAAAAAAGCAGGCTTCCTTGTCTACTCCACCCCCA CCCCC-3′ (SEQ ID NO:45) and oligo (reverse): 5′-GGGGACCACTTTGTACAAGAAAGCTGGGTCGTCATCCCATTCATCATCTTC ATC-3′ (SEQ ID NO:46) are used in the reaction.

2. The pcr fragment is cloned into pDONR201 vector (Invitrogen Life Technology, Cat# 11798-014) by Gateway BP reaction to generate pDONR WASP 105 L.

3. The pDONR WASP 105 L is cloned into pcDNA3.1MycTAP vector converted to Gateway destination vector by insertion a Gateway reading frame cassette.

D. Cloning of pcDNA3.1Myc_WASPTAP (Mammalian TAPTAG Tagged Protein)

1. WASP full length (American Type Culture Collection, Cat# 99534) is used as a template to amplify the coding sequence. Oligo (forward): 5′-GGGGACAAGTTTGTACAAAAAAGCAGGCTTCATGAGTGGGGGCCCAATG GGAGG-3′ (SEQ ID NO:47) and oligo (reverse): 5′-GGGGACCACTTTGTACAAGAAAGCTGGGTCGTCATCCCATTCATCATCTTC ATC-3′ (SEQ ID NO:46) are used in the reaction.

2. The pcr fragment is cloned into pDONR201 vector (Invitrogen Life Technology, Cat# 11798-014) by Gateway BP reaction to generate pDONR WASP fl.

3. The pDONR WASP fl is cloned into pcDNA3.1MycTAP vector converted to Gateway destination vector by inserting a Gateway reading frame cassette by Gateway LR reaction.

### EXAMPLE 2

### Cloning of N-WASP Proteins

A. Cloning of GST_N-WASPVCA

1. N-WASP full length cDNA is used as a template to amplify the coding sequence. Oligo (forward): 5′-GGGGACAAGTTTGTACAAAAAAGCAGGCTTCGAAAACCTGTATTTTCAGG GCTCTGATGGGGACCATCAG-3′ (SEQ ID NO:48) and oligo (reverse): 5′-GGGGACCACTTTGTACAAGAAAGCTGGGTCCTAGTCTTCCCACTCATCAT CATCCTC-3′ (SEQ ID NO:49) are used in the reaction.

2. The pcr fragment is cloned into pDONR201 (Invitrogen Life Technology, Cat# 11798-014) by Gateway BP reaction to generate pDONR_tev_HsN-WASPVCA.

3. Clone pDONR_tev_HsN-WASPVCA into pDEST15 (Invitrogen Life Technology, Cat# 11802-014) to generate N-GST_tev_HsN-WASPVCA by LR Gateway recombination reaction.

B. Cloning of N_GST_tev_{—}98FN-WASP (Bacterial GST Tagged Protein)

1. pENTR/SD/TOPO_N-WASP full length is used as a template to amplify the coding sequence. The oligo (forward): 5′-CACCGAAAACCTGTATTTTCAGGGCTTTGTATATAATAGTCCTAGAGGATA TTTTC-3′ (SEQ ID NO:50) and oligo (reverse): 5′-TTAGTCTTCCCACTCATCATCATC-3′ (SEQ ID NO:51) are used in the reaction.

2. The pcr fragment is cloned into /SD/TOPO vector (Invitrogen Life Technology, Cat# K2400-20) by directional cloning using Topoisomerase I.

3. The pENTR/SD/TOPO_tev_{—}98FN-WASP is cloned into pDEST15 (Invitrogen Life Technology, Cat# 11802-014) by Gateway LR reaction to generate N_GST_tev_{—}98FN-WASP.

C. Cloning of pcDNA3.1Myc_{—}98FN-WASPTAP (Mammalian TAPTAG Tagged Protein)

1. The pENTR/SD/TOPO_tev_{—}98FN-WASP is used as a template to amplify the coding sequence. Oligo (forward): 5′-GGGGACAAGTTTGTACAAAAAAGCAGGCTTCGAAAACCTGTATTTTCAGG GCTTTGTATATAATAGTCCTAGAGG-3′ (SEQ ID NO:52) and oligo (reverse): 5′-GGGGACCACTTTGTACAAGAAAGCTGGGTCGTCTTCCCACTCATCATCATC CTC-3′ (SEQ ID NO:53).

2. The pcr fragment is cloned into pDONR201 vector (Invitrogen Life Technology, Cat# 11798-014) by Gateway BP reaction to generate pDONR 98FN-WASP.

3. The pDONR 98FN-WASP is cloned into pcDNA3.1MycTAP vector converted to Gateway destination vector by insert a Gateway reading frame cassette by Gateway LR reaction.

D. Cloning of pcDNA3.1Myc_N-WASPTAP (Mammalian TAPTAG Tagged Protein)

1. HeLa total RNA is used as a template to amplify N-WASP by using SuperScript II RNase H-Reverse Transcriptase (Invitrogen Life Technology, Cat#18064-014). The oligo (forward): 5′-CACCGAAAACCTGTATTTTCAGGGCAGCTCCGTCCAGCAGCAGCCGCCG-3′ (SEQ ID NO:54) and oligo (reverse): 5′-TCAGTCTTCCCACTCATCATCATC-3′ (SEQ ID NO:55) are used in the reaction.

2. The pcr fragment is cloned into pENTR/SD/TOPO vector (Invitrogen Life Technology, Cat# K2400-20) by directional cloning using Topoisomerase I.

3. pENTR_N-WASP/SD/TOPO is used as a template to amplify the coding sequence. Oligo (forward): 5′-GCCGCTCGAGGTCTTCCCACTCATCATCATC-3′ (SEQ ID NO:56) and oligo (reverse): 5′-GCCGCTCGAGATGAGCTCCGTCCAGCAGC-3′ (SEQ ID NO:57) are used in the reaction.

4. The pcr fragment is digested with XhoI endonuclease and ligated into calf intestinal alkaline phosphatase (CIAP) treated pcDNA3.1MycTAP vector

5. Orientation of insert is checked to generate pcDNA3.1Myc_N-WASPTAP.

### EXAMPLE 3

### Cloning of Upstream Regulatory Proteins

A. Cloning of N_GST_tev_Cdc42 GTP (Bacterial GST Tagged Cdc42 Protein; SEQ ID NO:26)

1. pDONR_tev_Cdc42 wt is used as a template for QuickChange site-directed mutagenesis (Stratagene, Cat# 200518). Oligo(forward): 5′-TGTGTTGTTGTGGGCGATGTTGCTGTTGGTAAAACATGT-3′ (SEQ ID NO:58) and oligo(reverse): 5′-ACATGTTTTACCAACAGCAACATCGCCCACAACAACACA (SEQ ID NO:59) are used in this reaction to mutate G12 to a V.

2. Clone pDONR_tev_cdc42GTP into pDEST15 (Invitrogen Life Technology, Cat# 11802-014) to generate N-GST-tev-cdc42GTP by LR Gateway recombination reaction.

B. Cloning of N_GST_tev_RhoC GTP (Bacterial GST Tagged RhoC Protein; SEQ ID NO:28)

1. pDONR_tev_RhoC wt is used as a template for QuickChange site-directed mutagenesis (Stratagene, Cat# 200518). Oligo(forward): 5′-GTGATCGTTGGGGATGTTGCCTGTGGGAAGGAC-3′ (SEQ ID NO:60) and oligo(reverse): 5′-GTCCTTCCCACAGGCAACATCCCCAACGATCAC (SEQ ID NO:61) are used in this reaction to mutate G14 to a V.

2. Clone pDONR_tev_RhoC GTP into pDEST15 (Invitrogen Life Technology, Cat# 11802-014) to generate N-GST_tev_RhoC GTP by LR Gateway recombination reaction. An exemplary encoding sequence is SEQ ID NO:27.

C. Cloning of N_GST_tev_RhoA GTP (Bacterial GST Tagged RhoA Protein; SEQ ID NO:30)

1. RhoA GTP is used as a template to amplify the RhoA GTP coding sequence. Oligo (forward): 5′-GGGGACAAGTTTGTACAAAAAAGCAGGCTTCGAAAACCTGTATTTTCAGG GCGCTGCCATCCGGAAGAAACTGGTG-3′ (SEQ ID NO:62) and oligo (reverse):5′-GGGGACCACTTTGTACAAGAAAGCTGGGTCCTACAAGACAAGGCAACCAC ATTTTTTC-3′ (SEQ ID NO:63) are used in this reaction.

2. Clone pcr fragment into pDONR201 vector (Invitrogen Life Technology, Cat# 11798-014) by Gateway BP reaction to generate pDONR_tev_RhoA GTP.

3. Clone pDONR_tev_RhoAGTP into pDEST15 (Invitrogen Life Technology, Cat# 11802-014) to generate N-GST_tev_RhoA GTP by LR Gateway recombination reaction. An exemplary encoding sequence is SEQ ID NO:29.

D. Cloning of N_GST_tev_Rac1 GTP (Bacterial GST Tagged Rac1 Protein; SEQ ID NO:32)

1. Rac1 GTP is used as a template to amplify the Rac1 GTP coding sequence. Oligo (forward): 5′-GGGGACAAGTTTGTACAAAAAAACGGGCTTCGAAAACCTGTATTTTCAGG GCCAGGCCATCAAGTGTGTGGTGGTG-3′ (SEQ ID NO:64) and oligo (reverse): 5′-GGGGACCACTTTGTACAAGAAAGCTGGGTCCTACAACAGCAGGCATTTTC TCTTCCTC-3′ (SEQ ID NO:65) are used in this reaction.

2. Clone pcr fragment into pDONR201 vector (Invitrogen Life Technology, Cat# 11798-014) by Gateway BP reaction to generate pDONR_tev_Rac1 GTP.

3. Clone pDONR_{—tev}_Rac1 GTP into pDEST15 (Invitrogen Life Technology, Cat# 11802-014) to generate N-GST_tev_Rac1 GTP by LR Gateway recombination reaction. An exemplary coding sequence is SEQ ID NO:31.

E. Cloning of N_GST_tev_Nck1 (Bacterial GST Tagged Nck1 Protein; SEQ ID NO:34)

1. Nck cDNA (American Type Culture Collection, CAT# MGC-12668/4304621) is used as a template to amplify the Nck1 coding sequence. Oligo (forward): 5′-GGGGACAAGTTTGTACAAAAAAGCAGGCTTCGAAAACCTGTATTTTCAGG GCATGGCAGAAGAAGTGGTGGTAGTAG-3′ (SEQ ID NO:66) and oligo (reverse): 5′-GGGGACCACTTTGTACAAGAAAGCTGGGTCCTATGATAAATGCTTGACAA GATATAA-3′ (SEQ ID NO:67) are used in the reaction.

2. Clone pcr fragment into pDONR201 vector (Invitrogen Life Technology, Cat# 11798-014) by Gateway BP reaction to generate pDONR_tev_Nck1 GTP.

3. Clone pDONR_tev_Nck1 into pDEST15 (Invitrogen Life Technology, Cat# 11802-014) to generate N-GST_tev_Nck1 by LR Gateway recombination reaction. An exemplary coding sequence is SEQ ID NO:33.

F. Cloning of GST_NCK2 (SEQ ID NO:40)

1. NCK2 full length cDNA is used as a template to amplify the coding sequence. Oligo (forward): 5′-CACCATGACAGAAGAAGTTATTGTGATAGCC-3′ (SEQ ID NO:68) and oligo (reverse):5′-TCACTGCAGGGCCCTGACGAGGTAGAG-3′ (SEQ ID NO:69) are used in the reaction.

2. The pcr fragment is cloned into pENTR/SD/TOPO vector (Invitrogen Life Technology, Cat# K2400-20) by directional cloning using Topoisomerase I.

3. The pENTR/SD/TOPO_NCK2 is cloned into pDEST15 (Invitrogen Life Technology, Cat# 11802-014) by Gateway LR reaction to generate N_GST_NCK2. An exemplary coding sequence is SEQ ID NO:39.

### EXAMPLE 4

### Bacterial Expression of Fusion Proteins

Transformation: Competent cells (BL21(DE3) or BL21 STAR; Invitrogen) are thawed on ice and approximately 1 μl of DNA is added. Cells are gently mixed and incubated on ice for approximately 30 minutes. After heat shock at 42° C. for 45 seconds, cells are incubated on ice for 2 minutes and 0.5 ml SOC medium is added. Cells are allowed to recover by shaking at 37° C. for one hour, and then plated on selective media (typically LB+100 μg/ml ampicillin).

Day 1

For each new stock test for protein expression:
1. Inoculate several (2-4) 5-10 ml LB-Amp (75 μg/ml Ampicillin) cultures with small fractions of colonies. Mark colonies on a plate to be able to identify mother colony for each culture. Store plate at 4C. Grow inoculated cultures at 37° C. with shaking until OD₆₀₀=0.8-1. Remove 500 μl sample and collect cells by spinning the sample in an Eppendorf centrifuge 14 Krpm for 2 min; resuspend pellets in 100 μl SDS sample buffer.
2. Add IPTG to 0.5 mM to the remaining culture. Continue growing at 37° C. for 4 hours or at room temperature overnight.
3. Take another set of 500 μl gel samples: collect cells by spinning on an Eppendorf centrifuge 14 Krpm for 2 min; resuspend pellets in 100 μl SDS sample buffer; load 5 μl of each sample on a gel.

Day 2 (or 3)
1. Inoculate 250-500 ml of LB-Amp medium with a single tested colony.
2. Grow at 37° C. with shaking to OD₆₀₀˜0.6-0.8.
3. Collect cells by centrifugation on a table top centrifuge at 3 Krpm for 30 min.
4. Resuspend in 1/10 of initial volume in cold fresh LB-Amp/10% DMSO. Keep cell suspension on ice.
5. Pipette in 1 ml aliquots.
6. Freeze in LN₂. Store at −80° C.

### EXAMPLE 5

### Expression and Purification of Full Length WASP

TAPTAG WASP DNA is transfected using the Freestyle™ 293 expression system (Invitrogen Life Technologies, Cat K9000-01) in a scaled-up protocol:

A. Preparation of Cells for Transfection

(1) Freestyle™ 293-F cells are cultured in Freestyle™ culture medium according to manufacturer's directions (8% CO₂, 37° C.)

(2) Cells are split at 3×10⁵ cells/ml into 5×1000 ml sterile disposable PETG shaker flasks (Nalge Nunc Int, 4112-1000) with 0.45 μm vented closures (Nalge Nunc Int, 4114-0045) at 400 ml per flask and cultured on a shaking platform at 125 rpm for 96 hrs

(3) Cells are then expanded to 10×1000 ml shaker flasks (400 ml/flask) at 1.1×10⁶ cells/ml

B. Transfection of Cells

(1) Add 5.2 ml of 293 fectin™ to 140 ml of room temperature Opti-MEM® I reduced serum medium (Invitrogen Life Technologies, Cat 31985-070). Incubate at RT for 5 minutes

(2) Meanwhile, add 4 mg of pcDNA3.1_myc_TAP_WASP (prepared by QIAGEN Plasmid Giga Kit, Cat 12191) to 140 ml of room temperature Opti-MEM® I reduced serum medium

(3) Add the diluted DNA solution to the diluted 293 fectin™ solution and incubate at RT for 20 minutes

(4) Add 28 ml of this DNA/lipid mixture to each flask and then culture cells on a shaking platform at 125 rpm for 48 hrs

C. Preparation of Cells for TAPTAG WASP Purification

(1) Pool all flasks (to 4 liters total volume) and count cells

(2) Spin down cells (at 1500 rpm, 8 minutes, 4° C.) and resuspended in 1/10 volume (400 ml) ice cold PBS. Spin again (at 1500 rpm, 8 minutes, 4° C.) and freeze down cells in aliquots of 2.4×10⁹ cells in 50 ml sterile tubes using liquid nitrogen.

D. Purification of TAPTAG WASP

Cool down 500 ml of H₂O

RIPA FOR TAP-TAG STOCK 2×:

```
  10 mM TRIS pH 8.0
  2 mM EDTA
  2 mM EGTA
20% Glycerol
300 mM NaCl
```

Make 500 ml of the 2× buffer, filter and leave on 4° C.

To make 1× RIPA buffer just before using add:

| Final | | Stock | For 10 ml | | For 20 ml | |
|---|---|---|---|---|---|---|
| 1X | Stock | 2X | 5 | ml | 10 | ml |
| 1% | NP-40 | 20% | 500 | μl | 1 | ml |
| 0.125% | Deoxycholate | 5% | 0.5 | ml | 1 | ml |
| 1 mM | PMSF | 1M | 10 | μl | 20 | μl |
| | Inhibitors tablet | | 1 (small) | | 2 (small) | |
| 1 mM | Na₃VO₄ | 0.2 M | 50 | μl | 100 | μl |
| 1 mM | NaF | 0.5M | 20 | μl | 40 | μl |
| 20 mM | Beta glycerophosphate | | | | | |
| | H₂0 | | To 20 | ml | To 20 | ml |

To lyse cells: cover them with 1 ml of ice cold RIPA 1× buffer. Incubate them for 5 min, scrape them and leave for additional 25 min. Scrape again and transfer to cold 3 ml centrifuging tubes (Beckman). Wash plates with 0.2 ml RIPA 1× buffer and transfer solutions to the tubes. Spin for 66 Krpm 10 min (with 100 Krpm temperature rises).

Wash 400 μl (total) of IgG-Sepharose (Pharmacia) 4 times (4×10 ml) with IPP150:

| Final | | Stock | For 100 ml | |
|---|---|---|---|---|
| 10 mM | Tris-Cl pH8.0 | 1M stock | 1 | mL |
| 150 mM | NaCl | 5M | 3 | mL |
| 0.1% | NP40 | 20% | 0.5 | ml |
| | H₂0 | | To 100 | ml |

Pour cell lysate into 15 ml BIO-RAD column and add IgG resin. Shake for 2 h in cold room. Remove the top plug first, then the bottom plug and allow the column to drain by gravity flow.
Wash with 30 mL IPP150.
Wash with 10 mL TEV cleavage buffer.

TEV cleavage buffer:

| Final | | Stock | For 100 ml | |
|---|---|---|---|---|
| 10 mM | Tris-Cl pH8.0 | 1 M | 1 | mL |
| 150 mM | NaCl | 5 M | 3 | mL |
| 0.1% | NP40 | 20% | 0.5 | ml |
| 0.5 mM | EDTA | 0.5 M | 100 | μl |
| 1 mM | DTT | 1 M | 100 | μl |
| | H₂0 | | To 100 | ml |

Close the bottom of the column and add 1 ml of TEV buffer with 3 μl of TEV. protease (19 mg/ml). Shake for 1 h at RT.

Meanwhile, wash 200 μl of Calmodulin resin (Upstate) with CBB (Calmodulin binding buffer).

CBB—Calmodulin binding buffer:

| Final | | Stock | For 100 ml | |
|---|---|---|---|---|
| 10 mM | Tris-Cl pH8.0 | 1 M | 1 | mL |
| 150 mM | NaCl | 5 M | 3 | mL |
| 0.1% | NP40 | 20% | 0.5 | ml |
| 1 mM | MgCl₂ | 1 M | 100 | μl |
| 10 mM | BME (2- | 14.3 M | 69.9 | μl |
| | mercaptoethanol) | | | |
| 1 mM | Imidazole | 0.5 M | 200 | μl |
| 2 mM | CaCl₂ | 1 M | 200 | μl |
| | H₂0 | | To 100 | ml |

Remove the top and bottom plugs of the column and recover the eluate into the new 5 ml column by gravity flow. Elute the solution remaining in old column with an additional 300 μL of TEV cleavage buffer.

To the previous 1 mL eluate add:
3 volume of calmodulin binding buffer (3 mL) and
3 μL CaCl₂ 1M per mL of IgG eluate to titrate the EDTA coming from the TEV cleavage buffer.

After closing the column, rotate for 1 hour at 4° C. After binding allow the column to drain by gravity flow.
Wash with 30 mL CBB.

Elute 10 fractions of 100 μl with CEB calmodulin elution buffer. To elute add elution buffer ⅓ of the column volume, let the flow through come out. Close the column and incubate for 30 min. No shaking is required. Elute 10 100 μl fractions into siliconized tubes.

CEB-Calmodulin elution buffer:

| Final | | Stock | For 10 ml | |
|---|---|---|---|---|
| 10 mM | Tris-Cl pH8.0 | 1 M | 0.1 | mL |
| 150 mM | NaCl | 5 M | 0.3 | mL |
| 0.1% | NP40 | 20% | 50 | μl |
| 1 mM | MgCl₂ | 1 M | 10 | μl |
| 10 mM | BME (2- | 14.3 M | 7 | μl |
| | mercaptoethanol) | | | |
| 1 mM | Imidazole | 0.5 M | 20 | μl |
| 20 mM | EGTA | 0.25 M | 800 | μl |
| | H₂0 | | To 10 | ml |

Analogous procedures were utilized with TAPTAG N-WASP DNA, prepared as described in Example 2, to express and purify full length N-WASP.

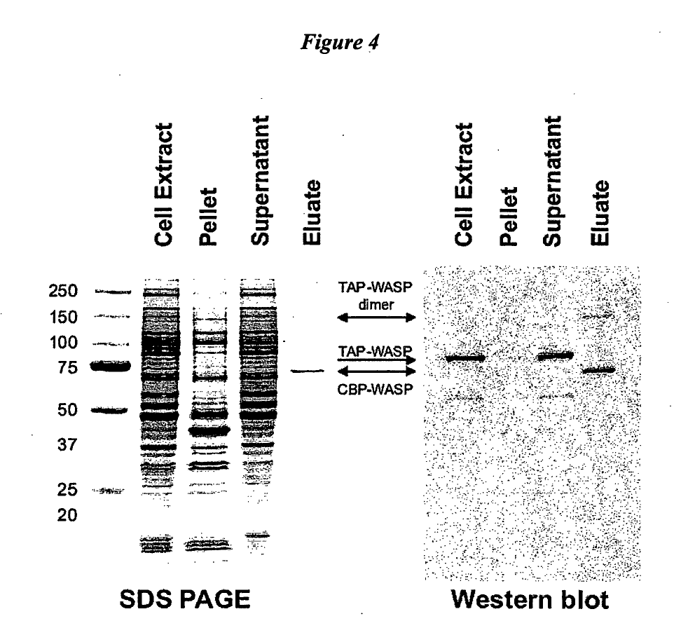

The full-length WASP or N-WASP produced according to the foregoing methods was at least 95% pure and was completely soluble. As shown in FIG. 4, no protein but WASP was observed in purified fractions.

### EXAMPLE 6

### Purification of Arp2/3 Complex

This example provides a description of an exemplary method for preparing purified Arp2/3 that can be utilized in the polymerization assays that are disclosed.

A. Materials

1. Buffer A:
10 mM Tris pH 8.0 (room temperature), 1 mM DTT, 1 mM MgCl, 30 mM KCl, 0.2 mM ATP, 1 mM EGTA KOH (0.25M stock pH 7) and 2% Glycerol

2. DEAE Buffer
Buffer A plus 2 tablets of protease inhibitors /1 and 1 mM PMSF.

3. Lysis Buffer:
50 mM Tris; 50 mM KCl; 10 mM Imidazole; 1 mM DTT; pH 7.0.

4. Tris Wash Buffer:
50 mM Tris; 50 mM KCl; 25 mM Imidazole, 1 mM DTT; pH 7.0.

5. Elution Buffer:
50 mM Tris; 300 mM Imidazole; 50 mM KCl; 1 mM DTT; pH 7.4

6. DEAE Chromatography Material (TOYOPEARL DEAE-650M; product #07473; manufactured by Tosh)

7. Q Sepharose Chromatography Material (Q Sepharose Fast Flow; product #17-0510-01, from Amersham Biosciences)

B. Preparation of Affinity Column Matrix

1. Synthesis and Expression of GST-VCA-His Fusion
WASP full length cDNA is used as a template to amplify the coding sequence. Oligo (forward): 5′-GGGGACAAGTTTGTACAAAAAAGCAGGCTTCGAAAACCTGTATTTTCAGGGCGG GGGTCGGGGAGCGCTTTTGGATC-3′ (SEQ ID NO:41) and oligo (reverse):5′-GGGGACCACTTTGTACAAGAAAGCTGGGTCCTAGTGATGGTGATGGTGATGGTA GTACGAGTCATCCCATTCATCATCTTCATC-3′ (SEQ ID NO:70) are used in the reaction.

The pcr fragment is cloned into pDONR201(Invitrogen Life Technology, Cat# 11798-014) by Gateway BP reaction to generate pDONR_tev_WASPVCA_His.

Clone pDONR_tev_WASPVCA_His into pDEST15 (Invitrogen Life Technology, Cat# 11802-014) to generate N-GST_tev_WASPVCA_His by LR Gateway recombination reaction.

The cloned DNA can be expressed as described in Example 4.

2. Purification of GST-VCA-His Fusion Protein

a. Growth conditions:
Inoculate culture in the morning with a single fresh colony (use B121(DE3)lysP cells). Use LB medium with (i.e. Sigma T-9179 or Gibco/BRL 22711-022) with 10 ppm antifoam.
Typical volume for a prep is 1-2 L. Use white baffled flask for 1 L of culture. Grow at 37° C. with shaking until OD₆₀₀ reaches 1.0-1.2. Shake at room temperature for 30-45 min. Add IPTG to 0.5 mM; continue shaking O/N.

b. Harvest cells following morning (after 12-16 hours) by spinning in a bench top Beckman centrifuge at 3 Krpm or in JLA 10 rotor at 5 Krpm for 30 minutes (4° C.).

From this point keep solutions on ice and/or at 4° C.

c. Resuspend cell pellets in Lysis buffer supplemented with 1× concentrations of Complete EDTA-free protease inhibitors (Boehringer 1836 170; use 1 mini-tablet per 10 ml) (20 ml for 1 L culture, 40 ml for 2 L). Use dounce homogenizer to make sure resuspension is complete. Proceed with a prep or freeze cell suspension in liquid N₂ and store at −80° C.

d. Cell disruption: When thawing cells add BME fresh. Lyze cells with the Microfluidizer by running 2 passes, 7-8 cycles each at 80 psi (on the green scale). (If using frozen cells, do 1 pass of 3 cycles). Pass some extra buffer (˜10 ml) through the chamber to rinse it.

e. Spin lysate in 45 Ti at 35 Krpm at 4° C. for 30 min. During this spin pre-equilibrate the resin with lysis buffer (see below).

f. Pre-equilibrate 1.5-2 ml (for 1 L culture) or 3 ml (for 2 L culture) of Ni-NTA resin (Qiagen cat. 31014) with Lysis buffer by washing 2 times with 15 ml of buffer without DTT and protease inhibitors. During these washes collect resin by spinning at 600-700 rpm for 2 min in a bench-top centrifuge.

g. Collect supernatant (save a sample for a gel). Batch load it onto Ni-resin. Incubate at 4° C. for 1 hr with rocking.

h. Pellet the resin by spinning at 600-700 rpm for 2 min. Decant supernatant (save sample for a gel). Resuspend in 5-10 ml of Lysis buffer (with BME and 1/10 of Complete inhibitors—i.e. 1 mini-tablet per 100 ml) and load resin into a column (use disposable columns or BioRad 1 cm ID EconoColumns). Wash with 50 ml of Lysis buffer. Washes can be done by gravity flow or with a peristaltic pump at 1 ml/min.

i. Pass 10 ml of Tris Wash Buffer through the column.

j. Elute with 8 1 ml fractions with Elution Buffer with 1/10 of protease inhibitors. Check protein concentrations in fractions by Coomassie Plus (Bradford). Pool peak fractions. (protein usually elutes starting at fraction 3).

Measure protein concentration in pooled fractions. Dilute with Tris Wash Buffer + 1/10 protease inhibitors to 2 mg/ml.

k. Freeze in liquid N₂ by “drop-freezing”. Store at −80° C.

3. Forming Affinity Matrix

The purified GST-VCA-His fusion is coupled to Glutathione-Sepharose (Amersham Biosciences) or related material according to the manufacturer's instructions.

C. Purification of Arp2/3

1. A cellular extract containing Arp2/3 complex was prepared from an Arp2/3 source such as human platelets (see, e.g., U.S. Provisional Application No. 60/578,969, filed Jun. 10, Welch and Mitchison (Meth. Enzymology 298:52-61, 1988), and Higgs, H. N., et al. (Biochemistry 38:15212-15222, 1999), all of which are incorporated herein by reference in their entirety for all purposes).

2. A DEAE column was packed with DEAE material and equilibrated with DEAE buffer. The amount of DEAE material included in the column was calculated based on 250 ml of resin for each 100 ml of crude extract.

3. The conductivity of the extract was adjusted to approximately 30 mM salt (3.6 mS is equivalent to 30 mM salt) and then loaded onto the DEAE column. Flowthrough was collected and the DEAE column washed with about 2 column volumes of DEAE buffer, which was also collected.

4. A Q-Sepharose column was packed and equilibrated with Buffer A. The amount of material was calculated based upon 100 ml of column material for each 200 ml of extract). The collected flowthrough and wash solution was loaded onto the equilibrated column. The column was then washed with 5-10 column volumes of Buffer A containing 30 mM KCl to displace proteins that did not bind or only loosely bound the column material. Bound proteins, including Arp2/3 complex, were subsequently eluted in Buffer A with a salt gradient of 30-300 mM KCl.

5. Fractions containing Arp2/3 were identified using the assay methods described herein and active fractions collected. The pooled fractions were diluted to obtain a conductivity of about 3.6 mS.

6. An affinity chromatography column containing the affinity matrix described above (i.e., GST-VCA-His6) was equilibrated in Buffer A. Pooled fractions enriched in Arp2/3 complex were then loaded onto the affinity column. The column was washed with about 5 volumes of Buffer A containing 30 mM KCl. Arp2/3 complex was eluted from the affinity column with 250 mM KCl in Buffer A.

7. Eluted fractions from the affinity column containing purified Arp2/3 were identified. Active fractions were concentrated in Y30 Centricons. The purified Arp2/3 was then diluted with fresh Buffer A to obtain a final solution containing about 30 mM KCl. Glycerol was added to about 30% (v/v) and the final protein solution stored at −20° C. The final protein had a purity of about 95% or more.

### EXAMPLE 7

### Actin Polymerization Protocol

A. Materials

G-Actin: Typically chicken actin was used. G-actin can be purchased from Cytoskeleton, Inc. It can also be purified according to Pardee and Spudich (1982) Methods of Cell Biol. 24:271-89, and subsequently gel filtered as discussed by MacLean-Fletcher and Pollard (1980) Biochem Biophys. Res. Commun. 96:18-27.

Pyrene-Actin: Typically chicken actin was utilized. Pyrene labeled actin was prepared according to methods described in Kouyama and Mihashi (1981) Eur. J. Biochem. 114:33-38 or as described by Cooper et al. (1983) J. Muscle Res. Cell Motility 4:253-62. Alternatively, it can be purchased from Cytoskeleton, Inc.

GST-Cdc42: Prepared as described in Examples 3 and 4.

GST-105 WASP: Prepared as described in Examples 1 and 4.

Arp2/3 Complex: Purified as described in Example 6.

Antifoam: Sigma antifoam

B. Concentration of Stock Reagents and Assay Composition

Arp2/3-mediated Actin Polymerization Protocol

| | | | Assay | |
|---|---|---|---|---|
| Reagents | Concentration | | Conc: | Unit |
| Actin | 0.8 | mg/ml | 3.41 | μM |
| Pyrene-actin | 1.5 | mg/ml | 0.55 | μM |
| GST-Cdc42 | 4.6 | mg/ml | 0.121 | μM |
| GST-105WASP | 0.2 | mg/ml | 0.044 | μM |
| Arp2/3 | 0.3 | mg/ml | 6.6 | nM |
| EGTA | 10 | mM | 55 | μM |
| Antifoam | 2% | | 22 | PPM |
| Number of plates | 35.00 | | | |
| Total Amount Needed | 397.00 | | | |
| First Step: Incubate | | | | |
| CDC-42 with GTP | | | | |
| Thaw appropriate amount ˜ | 588 | μl | | |

| and add GTP | | 65.3224638 | μl | Mix and keep at |
|---|---|---|---|---|
| | | | | room temperature |
| | | | | for 20 min |
| G-Buffer Total | | 265 | mls | Make G-buffer |
| | | | | on ice |
| 10× G-Buffer | | 27 | mls | |
| ATP | | 32 | mgs | Add fresh powder. |
| DTT | | 133 | μl | |
| Water | | 239 | mls | |
| Actin Mix (Mix 1) | Vol: | 223.5 | mls | Keep this mix on ice |

| G-buffer | 135.95 | mls | | |
|---|---|---|---|---|
| Actin | 80.02 | mls | 64.01934 | mgs |
| Pyrene-actin | 6.88 | mls | | |
| GST-Cdc42 | 587.90 | μL | | |
| Antifoam | 49.17 | μL | | |

| | | | |
|---|---|---|---|
| Arp2/3 Mix (Mix 2) | Vol: | 173.50 | mls |

| G-Buffer | 130 | mls | |
|---|---|---|---|
| GST-105WASP | 5344 | μL | |
| Arp2/3 | 1985 | μL | |
| Antifoam | 38 | μL | |
| EGTA | 1909 | μL | |
| 10× Polymerization Salts | 35 | mls | (add last, |
| | | | 400 mM KCl, |
| | | | 8 mM MgCl₂, |
| | | | 1× G-buffer w/o |
| | | | DTT, ATP) |

Samples containing candidate agents (individually or as mixtures) are placed into wells on a multi-well plate. Mix 1 is added to each of the wells and mixed with the candidate agent. A sample of Mix 2 is then introduced into each well and the resulting mixture thoroughly mixed. Typically, Mix 1 and Mix 2 are mixed in 1:1 ratio (e.g., 50 μl each of Mix 1 and Mix 2).

Actin polymerization is measured as a function of time by exciting pyrene at 365 nm and by detecting an increase in fluorescence emission at 407 nm. The change in fluorescence over time is utilized to determine a fluorescence parameter (e.g., maximal velocity, time to half maximal fluorescence intensity or area under the curve of a plot of fluorescence versus time).

### EXAMPLE 8

### Actin Polymerization Assay Using Full Length WASP

Full length WASP was prepared as described in Example 1. This protein was then used as a substitute GST-105WASP in methods that were otherwise identical to the methods described in Example 7.

### EXAMPLE 9

### Actin Polymerization Assay Using Full Length N-WASP

Full length N-WASP was prepared as described in Example 2. This protein was then used as a substitute GST-105 WASP in methods that were otherwise identical to the methods described in Example 7.

### EXAMPLE 10

### Evaluation of the Activity of Upstream Regulators on WASP and N-WASP Activity

A. Background

In this experiment, the in vitro pyrene-actin assay of the type described in Example 7 was utilized with full length human WASP and N-WASP to analyze the regulation of WASP and N-WASP by Cdc42, Rac1, RhoA, RhoC, Nck1, Nck2 and PIP₂.

B. Materials

Full length human WASP and N-WASP were TAP-tagged (Rigaut, et al. (1999) Nat. Biotechnology 17:1030, which is incorporated herein by reference in its entirety for all purposes) at the C-terminus (see, also Examples 1 and 2). The recombinant WASP and N-WASP were expressed in human 293 cells and then purified using a TAP-tag protocol as described in Example 5.

Arp2/3 was purified as described in Example 6.

Nck1, Nck2, Cdc42 and Rac1 were GST-tagged and purified as described in Examples 3 and 4 and then used in the assays.

C. Methods and Results

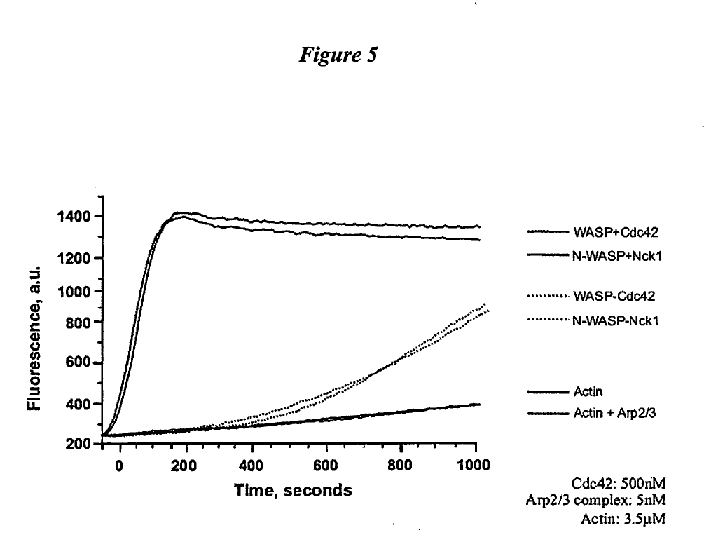

A first set of experiments were conducted to determine if full length WASP and N-WASP produced according to the methods described in Examples 1, 2 and 5 were regulated by upstream regulators such as Cdc42 and Nck1. Results are shown in FIG. 5. The activities shown this plot illustrate: 1) that FL-WASP and N-WASP by themselves could only weakly stimulate actin polymerization, and 2) that the upstream regulators or activators Cdc42 or Nck1 accelerated actin polymerization 13-fold. That FL WASP and N-WASP are regulated in a manner consistent with naturally occurring WASP and N-WASP indicates that the proteins produced by the methods provided herein are properly folded.

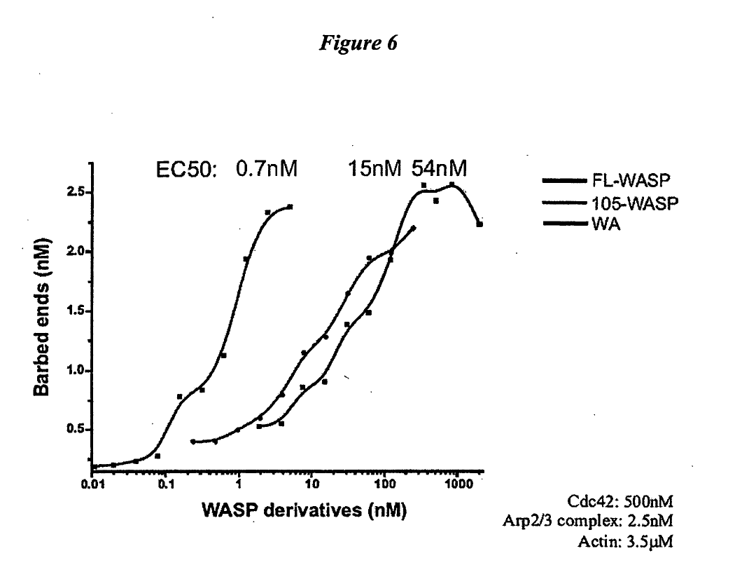

In a second set of experiments, the ability of various truncated forms of WASP were compared to the activity of the full-length protein. The polymerization assays were conducted in the presence of 500 nM Cdc42, 2.5 nm purified Arp2/3 complex and 3.5 μM actin. FL-WASP, 105 WASP and the VCA (see Example 7) domain were tested. The results of these trials were plotted to obtain EC50 values. The results are provided in FIG. 6 and in the chart below. These results demonstrate: 1) that at 3 nM, FL WASP stimulated production of maximal concentration of barbed ends, 2) that FL-WASP was approximately 20 times more active than 105 WASP, which lacks the WH1 domain, and 3) that FL WASP was more than 70 times more potent than the VCA/WA domain.

| | WASP | | | N-WASP | | |
|---|---|---|---|---|---|---|
| | | Barbed | | | Barbed | |
| | Barbed | ends | | Barbed | ends | |
| | ends, | % of | EC50, | ends, | % of | EC50, |
| Activator | nM* | max.** | nM | nM* | max.** | nM |
| Cdc42 | 3.8 | 87 | 16 | 1.3 | 9 | 287 |
| Rac1 | 1.4 | 12 | 80 | 1.9 | 28 | 31 |
| Nck1 | 4.0 | 94 | 10 | 3.4 | 75 | 11 |
| Nck2 | 2.6 | 50 | 12 | 3.5 | 78 | 7 |

*Total concentration of Arp2/3 complex in the assay is 4.2 nM
**After subtracting baseline (WASP without activators)

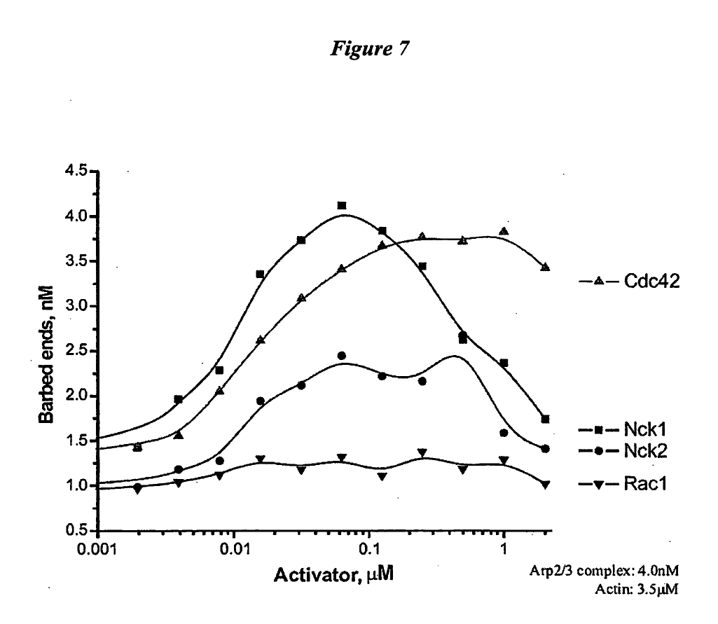

The ability of the upstream regulators Cdc42, Nck1, Nck2, and Rac1 to activate WASP was examined in a third experiment. The Arp2/3 complex in these experiments was 4.0 nM and the actin concentration 3.5 μM. The results are depicted in FIG. 7, which shows: 1) that Nck1 was the most potent of the activators tested, 2) that Cdc42 in the absence of Cdc42 can fully activate FL WASP, 3) that there is a bell shaped dependence between Nck1 and Nck2 and barbed end concentrations.

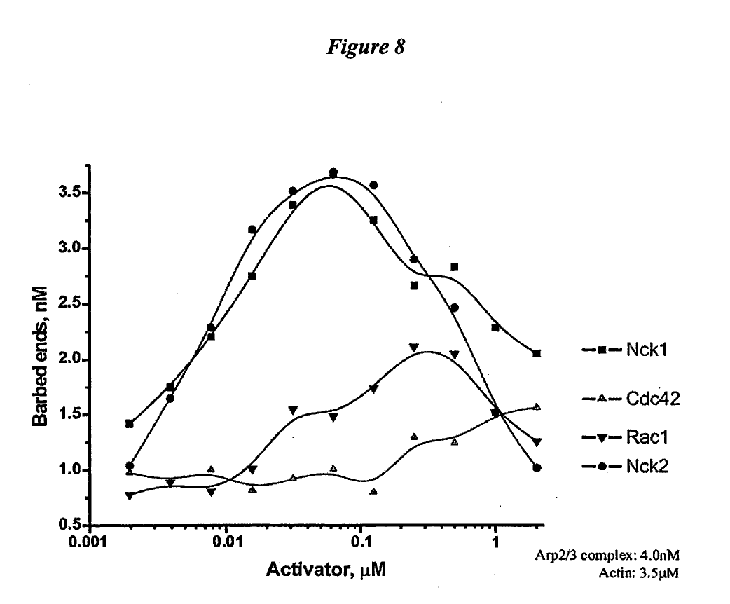

A fourth experiment similar to the third was conducted to ascertain the effect of Nck1, Nck2, Cdc42 and Rac1 on activation of FL N-WASP. Arp2/3 and actin concentrations were as described for the third experiment. FIG. 8 summarizes the results in graphical form and shows that: 1) Rac1 can activate FL N-WASP, 2) Rac1 was a more potent N-WASP activator than Cdc42 in the absence of PIP₂ vesicles, 3) Nck1 and Nck2 were the only activators tested that can stimulate production of maximal concentration of barbed ends; 4) Nck2 is a significantly better activator of N-WASP than WASP, and 5) there is a bell shaped dose dependence for Nck1, Nck2 and Rac1.

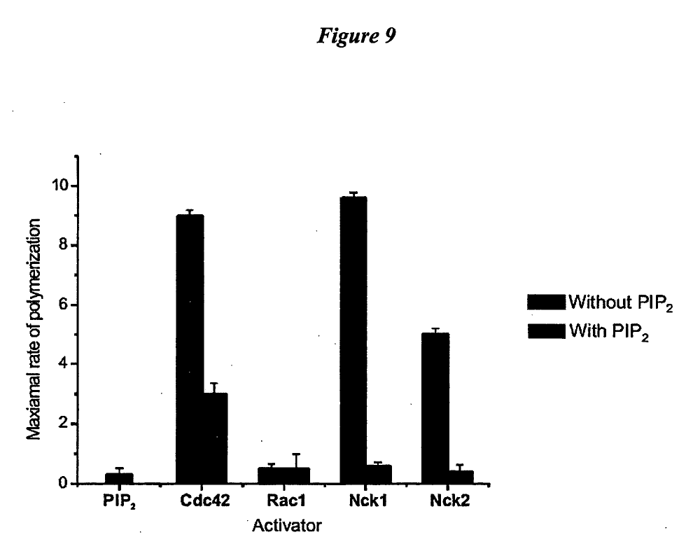

The effect of PIP₂on the ability of upstream regulators to regulate FL WASP was evaluated in a fifth set of experiments. The results are depicted in graphical format in FIG. 9. This figure indicates: 1) that PIP₂ had minimal, if any, effect of FL WASP in the absence of small GTPases or Nck, and 2) that PIP₂ had a strong inhibitory effect on WASP stimulated actin polymerization in the presence of both small GTPases or Nck.

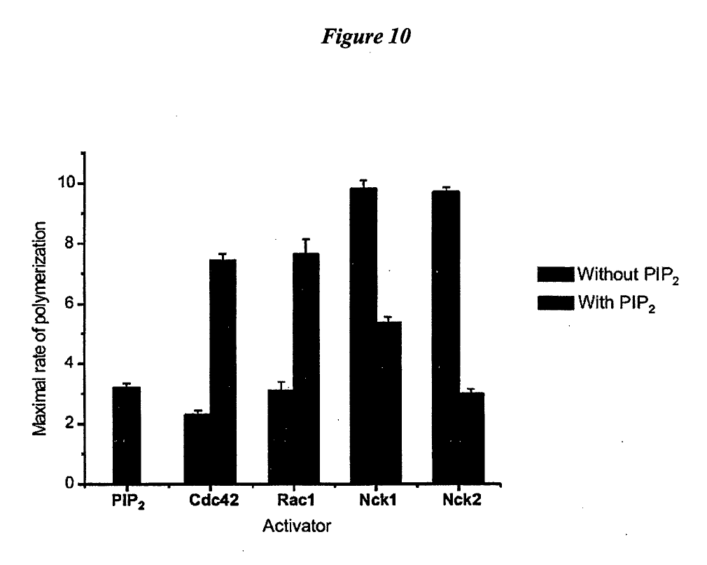

Another set of experiments similar to the fifth set were conducted using FL N-WASP. These results are shown in FIG. 10 and indicate: 1) that PIP₂ had a marked synergistic effect on N-WASP activation by Rac1 or Cdc42, and 2) PIP₂ inhibited Nck stimulated activation of N-WASP.

D. Conclusions

Some of the conclusions that can be drawn from the foregoing results are as follows:

1. Highly active an regulated recombinant FL WASP and N-WASP can be purified using the methods provided herein (see Example 5);

2. FL WASP was a more potent Arp2/3 complex activator than certain truncated derivatives such as 105 WASP and VCA.

3. Nck1 and Nck2 were the most powerful activators of FL WASP and FL N-WASP of the upstream regulatory proteins that were tested, as they stimulated generation of the maximal number of barbed ends.

4. Rac1 was a more potent FL N-WASP activator than Cdc42.

5. Cdc42 was more effective on WASP-stimulated actin nucleation by Arp2/3 complex than on N-WASP-stimulated actin nucleation.

6. At higher concentrations, Nck1, Nck2 and Rac1 inhibited WASP- and N-WASP-stimulated actin polymerization.

7. Lipid vesicles containing PIP₂ significantly improved actin nucleation by Arp2/3 complex and N-WASP in the presence of either of the small GTPases. In contrast, the vesicles had only a modest effect on WASP stimulated actin nucleation in the presence or absence of the GTPases.

8. PIP₂ had a strong inhibitory effect on WASP-stimulated actin polymerization.

9. PIP₂ had either a synergistically or an inhibitory effect on N-WASP activation by small GTPases or Nck, respectively.

10. In contrast to Rac1 and Cdc42, RhoA and RhoC could not activate either of the WASP family members.

Collectively, the results demonstrate that differential regulation of WASP and N-WASP by cellular activators reflects fundamental differences at the protein-protein level, and indicate that there are previously unrecognized regulatory interactions.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

TABLE 1
Approximate Boundaries of WASP and N-WASP domains (numbers in domain
columns refer to the amino acids of the corresponding SEQ ID NO:)

| | GenBank | | | | | | |
|---|---|---|---|---|---|---|---|
| | Accession | SEQ ID | WH-1 | B | CRIB/GBD | PolyPro | VCA |
| Protein | No. | NO: | Domain | Domain | Domain | Domain | Domain |
| WASP | P42768 | 2 | 1-142 | 219-237 | 230-288 | 312-421 | 429-501 |
| N-WASP | O00401 | 4 | 1-154 | 181-200 | 192-250 | 274-392 | 393-501 |

TABLE 2

| | SEQ ID NO: | | | |
|---|---|---|---|---|
| WASP/N- | (exemplary | SEQ ID NO: | | Regulated By Which |
| WASP Protein | nucleic acid) | (amino acid) | Activate Arp2/3? | Upstream Regulators? |
| FL-WASP | 1 | 2 | Yes | Cdc42, PIP₂, Nck and Rac1 |
| FL-N-WASP | 3 | 4 | Yes | Cdc42, PIP₂, Nck, Rac1 |
| WASP VCA | 5 | 6 | Yes | None |
| Domain | | | | |
| N-WASP VCA | 7 | 8 | Yes | None |
| Domain | | | | |
| 105WASP | 9 | 10 | Yes | Cdc42, PIP₂, Nck and Rac1 |
| 98 N-WASP | 11 | 12 | Yes | Cdc42, PIP₂, Nck and Rac1 |
| Myc-WASP- | 13 | 14 | Yes | Cdc42, PIP₂, Nck and Rac1 |
| TAP | | | | |
| Myc-N-WASP- | 15 | 16 | Yes | Cdc42, PIP₂, Nck and Rac1 |
| TAP | | | | |
| GST-105WASP | 17 | 18 | Yes | Cdc42, PIP₂, Nck and Rac1 |
| Myc-105WASP- | 19 | 20 | Yes | Cdc42, PIP₂, Nck and Rac1 |
| TAP | | | | |
| GST-tev-98N- | 21 | 22 | Yes | Cdc42, PIP₂, Nck and Rac1 |
| WASP | | | | |
| Myc-98N- | 23 | 24 | Yes | Cdc42, PIP₂, Nck and Rac1 |
| WASP-TAP | | | | |

What is claimed is:

1. A WASP protein analogue comprising in the amino terminal to carboxy terminal direction a B domain, a CRIB domain, and a VCA domain, wherein
the WH1 domain and/or PolyPro domain have been disabled; and
the WASP protein analogue can bind to an Arp2/3 complex and activate the nucleation activity of the Arp2/3 complex.

2. The WASP analogue of claim 1 that can be activated by an upstream regulator.

3. The WASP analogue of claim 2, wherein the upstream regulator is selected from the group consisting of Cdc42, phosphtidyl-1,4-bis phosphate (PIP₂), Nck1 and Rac1.

4. The WASP analogue of claim 3 that can be activated by Cdc42.

5. The WASP analogue of claim 1, wherein the WH1 domain is disabled.

6. The WASP analogue of claim 5 that comprises a WASP amino acid sequence in which amino acids 1 to 142 of SEQ ID NO:2 have been deleted.

7. The WASP analogue of claim 1, wherein the PolyPro domain is disabled.

8. The WASP analogue of claim 1 that comprises a WASP amino acid sequence in which amino acids 312-421 of SEQ ID NO:2 have been deleted.

9. The WASP analogue of claim 1, wherein the WH1 domain and the PolyPro domain are disabled.

10. The WASP analogue of claim 9 that comprises a WASP amino acid sequence in which amino acids 1 to 142 and amino acids 312-421 of SEQ ID NO:2 have been deleted.

11. The WASP analogue of claim 1 that comprises a WASP amino acid sequence in which
(i) amino acids 1-212 and amino acids 309-414 of SEQ ID NO:2 have been deleted;
(ii) amino acids 1-198 and amino acids 309-414 of SEQ ID NO:2 have been deleted;
(iii) amino acids 1-104 and amino acids 309-414 of SEQ ID NO:2 have been deleted; or
(iv) amino acid 1 and amino acids 309-414 of SEQ ID NO: 2 have been deleted.

12. The WASP analogue of claim 11 that is a fusion protein in which the WASP sequence is fused to a tag domain.

13. The WASP analogue of claim 12, wherein the tag is selected from the group consisting of: a TAP tag, a His tag, a glutathione-S-transferase (GST) tag, a calmodulin binding peptide (CBP) tag, an epitope tag, and a maltose-binding protein (MBP) tag.

14. The WASP analogue of claim 13, wherein the tag is a TAP tag.

15. A WASP protein analogue comprising in the amino terminal to carboxy terminal direction a B domain, a CRIB domain, a PolyPro domain and a VCA domain, wherein a segment of the WH1 domain has been deleted but the WASP protein analogue (i) can bind to an Arp2/3 complex and activate the nucleation activity of the Arp2/3 complex, and (ii) can be regulated by Cdc42, PIP₂, Nck and Rac1.

16. The WASP analogue of claim 15 that comprises a WASP amino acid sequence in which amino acids 1-104 of SEQ ID NO:2 have been deleted.

17. A recombinant or purified WASP protein that comprises the following characteristics:
(a) it comprises a WASP encoding segment that has at least 90% sequence identity to SEQ ID NO:2;
(b) it can be activated by Cdc42, PIP₂, Nck and Rac1; and
(c) it is soluble in aqueous solution.

18. The WASP protein of claim 17 that is a fusion protein that further comprises a carboxyl tag linked to the carboxyl end of the WASP encoding segment.

19. The WASP fusion protein of claim 18, wherein the carboxyl tag is a TAP tag that comprises in the amino terminal to carboxyl terminal direction a calmodulin binding peptide (CBP) domain, a TEV cleavage site, and a Prot A domain.

20. The WASP fusion protein of claim 19, further comprising an amino terminal tag linked to the amino terminal end of the WASP encoding segment.

21. The WASP fusion protein of claim 20, wherein the amino terminal tag is selected from the group consisting of: a myc tag, a His tag, a glutathione-S-transferase tag (GST), an epitope tag, and a maltose-binding protein (MBP) tag.

22. The WASP fusion protein of claim 21, wherein the amino terminal tag is a myc tag.

23. The WASP protein of claim 17, wherein the WASP encoding segment has at least 95% sequence identity to SEQ ID NO:2.

24. The WASP fusion protein of claim 22 that has the amino acid sequence of SEQ ID NO:14.

25. An N-WASP protein analogue comprising in the amino terminal to carboxy terminal direction a B domain, a CRIB domain, and a VCA domain, wherein
the WH1 domain and/or PolyPro domain have been disabled; and
the N-WASP protein analogue can bind to an Arp2/3 complex and activate the nucleation activity of the Arp2/3 complex.

26. The N-WASP analogue of claim 25, wherein the WH1 domain is disabled.

27. The N-WASP analogue of claim 25, wherein the PolyPro domain is disabled.

28. The N-WASP analogue of claim 25, wherein the WH1 domain and the PolyPro domain are disabled.

29. The N-WASP analogue of claim 25 that comprises an N-WASP sequence in which amino acids 1-97 of SEQ ID NO:2 have been deleted.

30. The N-WASP analogue of claim 29 that is a fusion protein in which the N-WASP sequence is fused to a tag domain.

31. The N-WASP analogue of claim 30, wherein the tag is selected from the group consisting of: a TAP tag, a His tag, a glutathione-S-transferase (GST) tag, a calmodulin binding peptide (CBP) tag, an epitope tag, and a maltose-binding protein tag.

32. An N-WASP analogue that (i) is a fragment of full length N-WASP (SEQ ID NO:4), (ii) comprises an amino acid sequence that has at least 90% sequence identity with respect to the full length of SEQ ID NO:12, and (iii) can bind to an Arp2/3 complex and activate the nucleation activity of the Arp2/3 complex.

33. The N-WASP analogue of claim 32 that is a fusion protein in which the amino acid sequence is fused to a tag.

34. The N-WASP analogue of claim 33, wherein the tag is selected from the group consisting of: a TAP tag, a His tag, a glutathione-S-transferase tag (GST), a calmodulin binding peptide (CBP) tag, an epitope tag, and a maltose-binding protein tag.

35. The N-WASP analogue of claim 34 that has the sequence of SEQ ID NO:12.

36. A recombinant or purified WASP protein that comprises the following characteristics:
(a) it comprises an N-WASP encoding segment that has at least 90% sequence identity to SEQ ID NO:4;
(b) it can be activated by Cdc42, PIP₂, Nck and Rac1; and
(c) it is soluble in aqueous solution.

37. The N-WASP protein of claim 36 that is a fusion protein that further comprises a carboxyl tag linked to the carboxyl end of the WASP encoding segment.

38. The N-WASP protein of claim 37, wherein the carboxyl tag is a TAP tag that comprises in the amino terminal to carboxyl terminal direction a calmodulin binding peptide (CBP) domain, a TEV cleavage site, and a Prot A domain.

39. The N-WASP protein of claim 38, further comprising an amino terminal tag linked to the amino terminus of the N-WASP encoding segment.

40. The WASP fusion protein of claim 39, wherein the amino terminal tag is selected from the group consisting of: a myc tag, a His tag, a glutathione-S-transferase tag (GST), an epitope tag, and a maltose-binding protein tag.

41. The N-WASP fusion protein of claim 40, wherein the amino terminal tag is a myc tag.

42. The N-WASP protein of claim 36, wherein the WASP encoding segment has at least 95% sequence identity to SEQ ID NO:4.

43. The N-WASP fusion protein of claim 41 that has the amino acid sequence of SEQ ID NO:16.

44. A nucleic acid construct encoding a WASP protein analogue comprising in the amino terminal to carboxy terminal direction a B domain, a CRIB domain, and a VCA domain, wherein
the WH1 domain and/or PolyPro domain have been disabled; and
the WASP protein analogue can bind to an Arp2/3 complex and activate the nucleation activity of the Arp2/3 complex.

45. A nucleic acid construct encoding a WASP protein analogue comprising in the amino terminal to carboxy terminal direction a B domain, a CRIB domain, a PolyPro domain and a VCA domain, wherein a segment of the WH1 domain has been deleted but the WASP protein analogue can (i) bind to an Arp2/3 complex and activate the nucleation activity of the Arp2/3 complex, and (ii) be regulated by Cdc42, PIP₂, Nck and Rac1.

46. A nucleic acid construct encoding a WASP protein, wherein the construct comprises a segment encoding a sequence with at least 90% sequence identity to SEQ ID NO:2 and a segment encoding a TAP tag that comprises a calmodulin binding domain and a ProtA binding domain, wherein the segments are operably linked.

47. The nucleic acid construct of claim 46 that encodes a protein comprising SEQ ID NO:14.

48. A nucleic acid construct encoding an N-WASP protein analogue comprising in the amino terminal to carboxy terminal direction a B domain, a CRIB domain, and a VCA domain, wherein
the WH1 domain and/or PolyPro domain have been disabled; and
the WASP protein analogue can bind to an Arp2/3 complex and activate the nucleation activity of the Arp2/3 complex.

49. A nucleic acid construct encoding an N-WASP analogue that (i) is a fragment of full length N-WASP (SEQ ID NO:4), (ii) has an amino acid sequence with at least 90% sequence identity with respect to the full length of SEQ ID NO:12, and (iii) can bind to an Arp2/3 complex and activate the nucleation activity of the Arp2/3 complex.

50. A nucleic acid construct encoding an N-WASP protein, wherein the construct comprises a segment encoding a sequence with at least 90% sequence identity to SEQ ID NO:4 and a segment encoding a TAP tag that comprises a calmodulin binding domain and a ProtA binding domain, wherein the segments are operably linked.

51. The nucleic acid construct of claim 50 that encodes a protein comprising SEQ ID NO:16.

52. A method of producing a WASP protein analogue, comprising expressing a nucleic acid construct of claim 44 in a host cell.

53. A method of producing an N-WASP protein analogue, comprising expressing a nucleic acid construct of claim 49 in a host cell.

54. A method of producing full-length WASP protein, comprising expressing a nucleic acid construct in a host cell, wherein the construct comprises a segment encoding a WASP protein and segment encoding a TAP tag that comprises a calmodulin binding domain and a Prot A binding domain, and wherein the WASP protein (i) has at least 90% sequence identity to SEQ ID NO:2, (ii) can be regulated by Cdc42, PIP₂, Nck, and Rac1, and (iii) is soluble in aqueous solution.

55. A method of producing full-length N-WASP protein, comprising expressing a nucleic acid construct in a host cell, wherein the construct comprises a segment encoding an N-WASP protein and segment encoding a TAP tag that comprises a calmodulin binding domain and a Prot A binding domain, and wherein the N-WASP protein (i) has at least 90% sequence identity to SEQ ID NO:4, (ii) can be regulated by Cdc42, PIP₂, Nck, and Rac1, and (iii) is soluble in aqueous solution.
